SEQUENCE LISTING (37 sequences)

SEQ ID NO: 1 - protein, 24 aa, Mumps virus
VLSIIAICLGSLGLILIILLSVVV

SEQ ID NO: 2 - protein, 21 aa, Mumps virus
WKLLTIVVANRNRMENFVYHK

SEQ ID NO: 3 - protein, 26 aa, Unknown
  PIV5 TMD
AIIVAALVLSILSIIISLLFCCWAYV

SEQ ID NO: 4 - protein, 28 aa, Unknown
  PIV5 CT
ATKEIRRINFKTNHINTISSSVDDLIRY

SEQ ID NO: 5 - protein, 713 aa, Artificial Sequence
  Fusion protein - MuV TMD and CT fused to a HIV antigen
MDRAKLLLLLLLLLLPQAQAVENLWVTVYYGVPVWKDAETTLFCASDAKAYDTEVRNVWATHACVPTDPNPQEIVLENVTENFNMWKNNMVEQMHTDIISLWDQSLKPCVKLTPLCVTLNCTNVNVTNTTNNTEEKGEIKNCSFNITTELRDKKKKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVVKKLREHFNNKTIIFNPSSGGDLEITTHSFNCGGEFFYCNTSGLFNSTWIGNGTKNNNNTNDTITLPCRIKQIINMWQRVGQPMYAPPIQGKIRCVSNITGLLLTRDGGNNNTNETETFRPGGGDMRDNWRSELYKYKVVKIEPLGVAPTRCKRRVVEGGGGSGGGGSAVGIGAVFLGFLGAAGSTMGAASMTLTVQARNLLSGGSGSGSGSTVWGIKQLQARVLAVERYLRDQQLLGIWGCSGKLICCTNVPWNSSWSNKSQDEIWDNMTWMEWDKEINNYTDIIYSLIEESQNQQEKNEQDLLALDKWASLWNWFDITNWLWYIKAIIVAALVLSILSIIISLLFCCWAYVATKEIRRINFKTNHINTISSSVDDLIRY

SEQ ID NO: 6 - DNA, 2142 bp, Artificial Sequence
  Fusion protein nucleotide sequence
atggacagagccaaactgctgctgctcctgttgctcctcctgctgcctcaggctcaggccgtggaaaatctgtgggtcaccgtgtactacggcgtgcccgtgtggaaggatgccgagacaacactgttctgtgccagcgacgccaaggcctacgataccgaagtgcggaatgtgtgggccactcacgcctgcgttcccaccgatcctaatcctcaagagatcgtgctggaaaacgtgaccgagaacttcaacatgtggaagaacaacatggtcgagcagatgcacaccgacatcatcagcctgtgggaccagagcctgaagccttgcgtgaagctgacccctctgtgcgtgaccctgaactgcaccaacgtgaacgtgaccaacaccaccaacaacaccgaggaaaagggcgagatcaagaactgcagcttcaacatcaccaccgagctgcgggacaagaaaaagaaggtgtacgccctgttctaccggctggacgtggtgcccatcgacgataacaacaacaactccagcaattaccggctgatcaactgcaacaccagcgccatcactcaggcctgtcctaaggtgtccttcgagcccattcctatccactactgtgcccctgccggcttcgccatcctgaagtgcaacgacaagaagttcaacggcacaggcccctgcaagaacgtgtccaccgtgcagtgtacccacggcatcaagccagtggtgtctacccagctgctgctgaatggctctctggccgaggaagagatcatcatcagaagcgagaacatcacgaacaacgccaagaccatcatcgtgcagctgaacgagagcgtggaaatcaattgcacccggcctaacaacaatacccggaagtccatcagaatcggccctggccagtggttttacgccaccggcgatatcatcggcgacatcagacaggcccactgtaacatcagcggcaccaagtggaacaagaccctgcagcaggtcgtgaagaagctgagagagcacttcaacaacaagacgatcatcttcaaccccagctctggcggcgacctggaaatcaccacacacagcttcaattgtggcggcgagttcttctactgcaatacctccggcctgttcaacagcacctggatcggcaatggcaccaagaacaacaacaacaccaacgacaccatcacactgccctgccggatcaagcagatcatcaatatgtggcagcgcgtgggccagcctatgtacgctcctccaatccagggcaagatcagatgcgtgtccaatatcaccggcctgctgctcacaagagatggcggaaacaacaacacgaatgagacagagacattcagacccggcggaggcgacatgagagacaattggagaagcgagctgtacaagtacaaggtggtcaagatcgagcccctgggcgtcgcacctacacggtgcaaaagaagagtggtcgaaggcggcggaggaagcggaggcggaggatctgctgttggaatcggagccgtgttcctgggctttctgggagccgctggatctacaatgggcgctgccagcatgaccctgacagtgcaggctagaaatctgctgtctggcggcagcggctctggctcaggatctacagtgtggggaatcaagcagctgcaggccagagtgctggccgtcgagagatacctgagagatcagcagctcctcggcatctggggctgttctggcaagctgatctgctgcaccaatgtgccctggaacagctcctggtccaacaagagccaggacgagatctgggacaacatgacctggatggaatgggacaaagagattaacaactacacggatatcatctacagcctgatcgaggaaagccagaaccagcaagagaagaacgagcaggacctgctggccctggataagtgggcttccctgtggaattggttcgacatcaccaactggctgtggtacatcaaggccatcattgtggccgctctggtgctgagcatcctgtccatcatcatctccctgctgttctgctgctgggcctacgtggccaccaaagagatcagacggatcaacttcaagaccaaccacatcaacaccatcagctccagcgtggacgacctgatccggtactag

SEQ ID NO: 7 - protein, 704 aa, Artificial Sequence
  Fusion protein - PIV5 TMD and CT fused to HIV antigen
MDRAKLLLLLLLLLLPQAQAVENLWVTVYYGVPVWKDAETTLFCASDAKAYDTEVRNVWATHACVPTDPNPQEIVLENVTENFNMWKNNMVEQMHTDIISLWDQSLKPCVKLTPLCVTLNCTNVNVTNTTNNTEEKGEIKNCSFNITTELRDKKKKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVVKKLREHFNNKTIIFNPSSGGDLEITTHSFNCGGEFFYCNTSGLFNSTWIGNGTKNNNNTNDTITLPCRIKQIINMWQRVGQPMYAPPIQGKIRCVSNITGLLLTRDGGNNNTNETETFRPGGGDMRDNWRSELYKYKVVKIEPLGVAPTRCKRRVVEGGGGSGGGGSAVGIGAVFLGFLGAAGSTMGAASMTLTVQARNLLSGGSGSGSGSTVWGIKQLQARVLAVERYLRDQQLLGIWGCSGKLICCTNVPWNSSWSNKSQDEIWDNMTWMEWDKEINNYTDIIYSLIEESQNQQEKNEQDLLALDKWASLWNWFDITNWLWYIKVLSIIAICLGSLGLILIILLSVVVWKLLTIVVANRNRMENFVYHK

SEQ ID NO: 8 - DNA, 3354 bp, Artificial Sequence
  Fusion protein nucleotide sequence
atggacagagccaaactgctgctgctcctgttgctcctcctgctgcctcaggctcaggccgtggaaaatctgtgggtcaccgtgtactacggcgtgcccgtgtggaaggatgccgagacaacactgttctgtgccagcgacgccaaggcctacgataccgaagtgcggaatgtgtgggccactcacgcctgcgttcccaccgatcctaatcctcaagagatcgtgctggaaaacgtgaccgagaacttcaacatgtggaagaacaacatggtcgagcagatgcacaccgacatcatcagcctgtgggaccagagcctgaagccttgcgtgaagctgacccctctgtgcgtgaccctgaactgcaccaacgtgaacgtgaccaacaccaccaacaacaccgaggaaaagggcgagatcaagaactgcagcttcaacatcaccaccgagctgcgggacaagaaaaagaaggtgtacgccctgttctaccggctggacgtggtgcccatcgacgataacaacaacaactccagcaattaccggctgatcaactgcaacaccagcgccatcactcaggcctgtcctaaggtgtccttcgagcccattcctatccactactgtgcccctgccggcttcgccatcctgaagtgcaacgacaagaagttcaacggcacaggcccctgcaagaacgtgtccaccgtgcagtgtacccacggcatcaagccagtggtgtctacccagctgctgctgaatggctctctggccgaggaagagatcatcatcagaagcgagaacatcacgaacaacgccaagaccatcatcgtgcagctgaacgagagcgtggaaatcaattgcacccggcctaacaacaatacccggaagtccatcagaatcggccctggccagtggttttatgccaccggcgatattatcggcgacatcagacaggcccactgtaacatcagcggcaccaagtggaacaagaccctgcagcaggtcgtgaagaagctgagagagcacttcaacaacaagacgatcatcttcaaccccagctctggcggcgacctggaaatcaccacacacagcttcaattgtggcggcgagttcttctactgcaatacctccggcctgttcaacagcacctggatcggcaatggcaccaagaacaacaacaacaccaacgacaccatcacactgccctgccggatcaagcagatcatcaatatgtggcagcgcgtgggccagcctatgtacgctcctccaatccagggcaagatcagatgcgtgtccaatatcaccggcctgctgctcacaagagatggcggaaacaacaacacgaatgagacagagacattcagacccggcggaggcgacatgagagacaattggagaagcgagctgtacaagtacaaggtggtcaagatcgagcccctgggcgtcgcacctacacggtgcaaaagaagagtggtcgaaggcggcggaggaagcggaggcggaggatctgctgttggaatcggagccgtgttcctgggctttctgggagccgctggatctacaatgggcgctgccagcatgaccctgacagtgcaggctagaaatctgctgtctggcggcagcggctctggctcaggatctacagtgtggggaatcaagcagctgcaggccagagtgctggccgtcgaaagatacctgagagatcagcagctcctcggcatctggggctgttctggcaagctgatctgctgcaccaatgtgccctggaacagctcctggtccaacaagagccaggacgagatctgggacaacatgacctggatggaatgggacaaagagattaacaactatacggacatcatctacagcctgatcgaggaaagccagaaccagcaagagaagaacgagcaggacctgctggccctggataagtgggcttccctgtggaattggttcgacatcaccaactggctgtggtacatcaaggtgctgagcatcattgccatctgcctgggcagcctgggcctgatcctgatcattctgctgagcgtggtcgtgtggaaactgctgacaatcgtggtggccaaccggaaccggatggaaaacttcgtgtaccacaagcggcgcagaaggcggagaggatctggcgaaggcagaggctctctgctgacatgtggcgacgtggaagagaaccctggacctatgggatgcgtgcagtgcaaggacaaagaacccagcatcagcatccccgccgatcctacaaaccccagacagagcatcaaggcctttccaatcgtgatcaacagcgacggcggcgagaagggcagactggttaagcagctgagaaccacctacctgaacgacctggacacccacgagcctctggtcaccttcgtgaacacctacggcttcatctacgaacaggaccggggcaacacaatcgtcggcgaagatcagctgggcaagaaacgggaagccgtgacagccgccatggtcacacttggctgtggccctaatctgcctagcctgggcaatgtgcttggccagctgagcgagttccaagtgattgtgcgcaagaccagcagcaaggccgaagagatggtgttcgagatcgtgaagtaccccagaatcttccggggccacacactgatccagaaaggcctcgtgtgtgtgtccgccgagaagttcgtgaagtctcccggcaaggtgcagagcggcatggactacctgttcatccccacctttctgagcgtgacctactgtcctgccgccatcaagttccaggtgccaggacctatgctgaagatgcggagcagatacacccagtctctgcagctggaactgatgatcagaatcctgtgcaagcccgacagtcccctgatgaaggtgcacatccccgacaaagaaggcaggggctgtctcgtgtctgtgtggctgcacgtgtgcaacatcttcaagagcggcaacaagaacggcagcgagtggcaagagtactggatgcggaagtgcgccaacatgcagctcgaagtgtctatcgccgacatgtggggccctaccatcatcatccacgccagaggacacatccccaagagcgccaagctgttctttggcaaaggcggctggtcctgccatcctctgcatgaggttgtgcccagcgtgaccaagacactttggagcgtgggctgcgagatcaccaaggccaaggccattatccaagagagcagcatctccctgctggtggaaaccacagacatcattagccccaaagtgaagatctccagcaagcacagaagattcggcaagagcaactggggcctgtttaaaaagaccaagagcctgcctaacctgaccgagctggaatag

SEQ ID NO: 9 - protein, 10 aa, Unknown
  MTS derived from a Fyn-like protein kinase
MGCVQCKDKE

SEQ ID NO: 10 - protein, 10 aa, Unknown
  MTS derived from a Lck-M
MGCGCSSHPE

SEQ ID NO: 11 - protein, 10 aa, Unknown
  MTS derived from Src
MGSSKSKPKD

SEQ ID NO: 12 - protein, 10 aa, Unknown
  MTS derived from C-YES
MGCIKSKENK

SEQ ID NO: 13 - protein, 384 aa, Artificial Sequence
  Fusion protein - MuV matrix protein and a MTS derived from a Fyn-like protein kinase
MGCVQCKDKEAGSQIKIPLPKPPDSDSQRLNAFPVIMAQEGKGRLLRQIRLRKILSGDPSDQQITFVNTYGFIRATPETSEFISESSQQKVTPVVTACMLSFGAGPVLEDPQHMLKALDQTDIRVRKTASDKEQILFEINRIPNLFRHHQISADHLIQASSDKYVKSPAKLIAGVNYIYCVTFLSVTVCSASLKFRVARPLLAARSRLVRAVQMEVLLRVTCKKDSQMAKSMLNDPDGEGCIASVWFHLCNLCKGRNKLRSYDENYFASKCRKMNLTVSIGDMWGPTILVHAGGHIPTTAKPFFNSRGWVCHPIHQSSPSLAKTLWSSGCEIKAASAILQGSDYASLAKTDDIIYSKIKVDKDAANYKGVSWSPFRKSASMSNL

SEQ ID NO: 14 - DNA, 1155 bp, Artificial Sequence
  Fusion protein nucleotide sequence comprising a Fyn proto-oncogene sequence
atgggctgtgtgcaatgtaaggataaagaagctggatcacagatcaaaattcctcttccaaagccccccgattcagactctcaaagattaaatgcattccctgtaatcatggctcaagaaggcaaaggacgactcctcagacaaatcagacttaggaaaatattatcaggggatccatccgatcagcaaatcacattcgtgaatacatatggattcatccgtgccactccagaaacgtccgagttcatctctgaatcatcacaacaaaaggtgactcctgtagtgacggcgtgtatgctgtccttcggtgctggaccagtcctagaagacccacaacatatgctgaaagctcttgatcagacagatatcagggttcggaagacagcgagtgacaaagagcagatcttattcgagatcaaccgcatccccaatctattcaggcatcatcaaatatctgcggaccatctgattcaggccagttccgataaatatgtcaagtcaccagcaaagttgattgcaggagtaaattacatctactgtgtcacatttttatccgtgacagtttgttccgcctcactcaaatttcgggttgcgcgcccattgcttgctgcacgatctagattagtaagagcagttcagatggaagttttgcttcgggtaacttgcaaaaaagactcccaaatggcaaagagcatgttaaatgaccctgatggagaagggtgcattgcatccgtgtggttccacctgtgtaatctgtgcaaaggcaggaataaacttagaagttatgatgaaaattattttgcatccaagtgccgtaagatgaacctgacagtcagcataggagacatgtggggaccaaccattctagtccatgcaggcggtcatattccgacaactgcaaaaccctttttcaactcaagaggctgggtttgccaccccatccaccaatcatcaccatcgttggcgaagaccctatggtcatctgggtgtgaaatcaaggctgccagtgctatcctccagggctcagactatgcatcacttgcaaaaactgatgacataatatattcaaagataaaagttgataaagatgcagccaactacaaaggagtatcctggagtccattcaggaagtctgcctcaatgagcaacctatga

SEQ ID NO: 15 - DNA, 1155 bp, Artificial Sequence
  Fusion protein nucleotide sequence
atgggctgcgtgcagtgcaaggacaaagaggccggcagccagatcaagatccccctgcccaagccccccgacagcgatagccagagactgaacgccttccccgtgatcatggcccaggaaggcaagggcagactgctgcggcagatccggctgagaaagatcctgagcggcgaccccagcgaccagcagatcaccttcgtgaacacctacggcttcatccgggccacccccgagacaagcgagttcatcagcgagagcagccagcagaaagtgacccccgtcgtgaccgcctgcatgctgtcttttggagccggccctgtgctggaagatccccagcacatgctgaaggccctggaccagaccgacatcagagtgcgcaagaccgccagcgacaaagagcagatcctgttcgagatcaaccgcatccccaacctgttccggcaccaccagatcagcgccgaccacctgattcaggccagctccgacaaatacgtgaagtcccccgccaagctgatcgccggcgtgaactatatctactgcgtgaccttcctgagcgtgaccgtgtgcagcgccagcctgaagttcagagtggccagacctctgctggccgccagatctagactcgtgcgggccgtgcagatggaagtgctgctgagagtgacctgcaagaaagacagccagatggccaagagcatgctgaacgaccccgacggcgagggctgtatcgccagcgtgtggttccacctgtgcaatctgtgcaagggccggaacaagctgcggagctacgacgagaactacttcgccagcaagtgccggaagatgaacctgaccgtgtccatcggcgacatgtggggccctaccatcctggtgcatgccggcggacacatccctaccaccgccaagccattcttcaacagccggggctgggtgtgccaccccatccatcagtctagccccagcctggccaagaccctgtggtctagcggctgcgagatcaaggccgcctctgccatcctgcagggcagcgattatgcctccctggccaaaaccgacgacatcatctacagcaagatcaaggtggacaaggacgccgccaactacaagggagtgtcttggagccccttcagaaagtccgccagcatgagcaacctgtaa

SEQ ID NO: 16 - DNA, 1155 bp, Artificial Sequence
  Fusion protein nucleotide sequence
atgggctgtgtgcagtgcaaggacaaagaggccggcagccagatcaagatccctctgcctaagcctcctgacagcgacagccagagactgaacgcctttcctgtgatcatggcccaagaaggcaagggcagactgctgcggcagatccggctgagaaagatcctgagcggcgaccctagcgaccagcagatcaccttcgtgaacacctacggcttcatccgggccacacctgagacaagcgagttcatcagcgagagcagccagcagaaagtgacccctgtggtcaccgcctgcatgctgtcttttggagccggacctgtgctggaagatccccagcacatgctgaaggccctggaccagaccgacatcagagtgcggaaaaccgccagcgacaaagagcagatcctgttcgagatcaacagaatccccaacctgttccggcaccaccagatctctgccgaccatctgattcaggccagctccgacaaatacgtgaagtcccctgccaagctgatcgccggcgtgaactatatctactgcgtgaccttcctgagcgtgaccgtgtgtagcgccagcctgaagttcagagtggccagacctctgctggccgccagaagcagacttgttagagccgtgcagatggaagtgctgctgagagtgacctgcaagaaagactcccagatggccaagagcatgctgaacgaccctgatggcgagggctgtatcgccagcgtgtggttccacctgtgcaatctgtgcaaaggccggaacaagctgcggagctacgacgagaattacttcgccagcaagtgccggaagatgaacctgaccgtgtccatcggcgatatgtggggccctacaatcctggtgcatgccggcggacacatccctacaaccgccaagccattcttcaactccagaggctgggtctgccatcctatccaccagtctagccccagcctggccaagacactttggagcagcggatgcgagatcaaggccgcctctgctatcctgcagggcagcgattatgcctctctggccaaaaccgacgacatcatctacagcaagatcaaggtggacaaggacgccgccaactacaagggagtcagctggtccccattccggaagtctgccagcatgagcaacctgtaa

SEQ ID NO: 17 - protein, 386 aa, Artificial Sequence
  Fusion protein - PIV5 matrix protein and a MTS derived from a Fyn-like protein kinase
MGCVQCKDKEPSISIPADPTNPRQSIKAFPIVINSDGGEKGRLVKQLRTTYLNDLDTHEPLVTFVNTYGFIYEQDRGNTIVGEDQLGKKREAVTAAMVTLGCGPNLPSLGNVLGQLSEFQVIVRKTSSKAEEMVFEIVKYPRIFRGHTLIQKGLVCVSAEKFVKSPGKVQSGMDYLFIPTFLSVTYCPAAIKFQVPGPMLKMRSRYTQSLQLELMIRILCKPDSPLMKVHIPDKEGRGCLVSVWLHVCNIFKSGNKNGSEWQEYWMRKCANMQLEVSIADMWGPTIIIHARGHIPKSAKLFFGKGGWSCHPLHEVVPSVTKTLWSVGCEITKAKAIIQESSISLLVETTDIISPKVKISSKHRRFGKSNWGLFKKTKSLPNLTELE

SEQ ID NO: 18 - DNA, 1161 bp, Artificial Sequence
  Fusion protein nucleotide sequence
atgggctgtgtgcaatgtaaggataaagaaccatccatcagcatccccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcgtaaatacctatggattcatctacgaacaggatcgggggaatactattgtcggagaggatcaacttgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggctgcatgtatgcaacatcttcaaatcaggaaacaagaatggcagtgagtggcaggaatactggatgagaaagtgtgccaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttatcgtggagactactgacatcataagtccaaaagtcaaaatttcatctaagcatcgccgctttgggaaatcaaattggggtctgttcaagaaaactaaatcactgcctaacctgacggagctggaatga

SEQ ID NO: 19 - DNA, 1161 bp, Artificial Sequence
  Fusion protein nucleotide sequence
atgggctgcgtgcagtgcaaggacaaagagcccagcatcagcatccccgccgaccccaccaatccccggcagagcattaaggccttccccatcgtgatcaacagcgacggcggcgagaagggccggctcgtgaaacagctgagaaccacctacctgaacgacctggacacccacgagcccctcgtgaccttcgtgaacacctacggcttcatctacgagcaggaccggggcaacaccatcgtgggcgaagatcagctgggcaagaaacgcgaggccgtgacagccgccatggtcacactgggctgtggccccaatctgccctccctgggaaatgtgctgggccagctgagcgagttccaagtgatcgtgcgcaagaccagcagcaaggccgaggaaatggtgttcgagatcgtgaagtacccccggatcttccggggccacaccctgatccagaaaggcctcgtgtgtgtgtccgccgagaagtttgtgaagtcccctggcaaggtgcagagcggcatggactacctgttcatccccacctttctgagcgtgacctactgccctgccgccatcaagttccaggtgccaggccccatgctgaagatgcggagcagatacacccagagcctgcagctggaactgatgatcagaatcctgtgcaagcccgacagccccctgatgaaggtgcacatccccgacaaagagggcagaggctgcctggtgtctgtgtggctgcacgtgtgcaacatcttcaagagcggcaacaagaacggcagcgagtggcaggaatactggatgcggaagtgcgccaacatgcagctggaagtgtctatcgccgacatgtggggccctaccatcatcatccacgccagaggccacatccccaagagcgccaagctgttctttggcaagggcggctggtcctgccaccctctgcatgaggtggtgccctccgtgaccaagaccctgtggagcgtgggctgcgagatcaccaaggccaaggccatcatccaggaaagcagcatctccctgctggtggaaaccaccgacatcatcagccccaaagtgaagatctccagcaagcacagaagattcggcaagagcaactggggcctgttcaaaaagaccaagagcctgcccaacctgaccgagctggagtaa

SEQ ID NO: 20 - DNA, 1161 bp, Artificial Sequence
  Fusion protein nucleotide sequence
atgggctgtgtgcagtgcaaggacaaagagcccagcatcagcatccccgccgatcctaccaatcctcggcagagcatcaaggcctttccaatcgtgatcaacagcgacggcggcgagaagggcagactggttaagcagctgagaaccacctacctgaacgacctggacacccacgagcctctggtcaccttcgtgaacacctacggcttcatctacgagcaggaccggggcaataccatcgtgggcgaagatcagctgggcaagaaacgggaagccgtgacagccgccatggtcacacttggctgtggccctaatctgcctagcctgggcaatgtgctgggccagctgagcgagttccaagtgatcgtgcggaaaaccagcagcaaggccgaggaaatggtgttcgagatcgtgaagtaccccagaatcttccggggccacacactgatccagaaaggcctcgtgtgtgtgtccgccgagaagttcgtgaagtctcccggcaaggtgcagagcggcatggactacctgttcatccccacctttctgagcgtgacctactgtcctgccgccatcaagttccaggtgccaggacctatgctgaagatgcggagcagatacacacagagcctgcagctggaactgatgatcagaatcctgtgcaagccagacagccctctgatgaaggtgcacatccccgacaaagaaggcagaggctgcctggtgtctgtgtggctgcacgtgtgcaacatcttcaagagcggcaacaagaacggcagcgagtggcaagagtactggatgcggaagtgcgccaacatgcagctcgaagtgtctatcgccgacatgtggggccctaccatcatcatccacgccagaggacacatccccaagagcgccaagctgttctttggcaaaggcggctggtcctgccatcctctgcatgaggttgtgcccagcgtgaccaagacactttggagcgtgggctgcgagatcaccaaggccaaggccatcatccaagagagcagcatctccctgctggtggaaaccaccgacatcatcagccccaaagtgaagatctccagcaagcacagaagattcggcaagagcaactggggcctgttcaaaaagaccaagagcctgcctaacctgaccgagctggaataa

SEQ ID NO: 21 - protein, 27 aa, Unknown
  Furin/T2A
RRRRRRGSGEGRGSLLTCGDVEENPGP

SEQ ID NO: 22 - protein, 1124 aa, Artificial Sequence
  Fusion protein - MuV matrix protein, a MTS derived from a Fyn-like protein kinase, a MuV TMD and CT fused to HIV antigen and a furin/T2A sequence
MDRAKLLLLLLLLLLPQAQAVENLWVTVYYGVPVWKDAETTLFCASDAKAYDTEVRNVWATHACVPTDPNPQEIVLENVTENFNMWKNNMVEQMHTDIISLWDQSLKPCVKLTPLCVTLNCTNVNVTNTTNNTEEKGEIKNCSFNITTELRDKKKKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVVKKLREHFNNKTIIFNPSSGGDLEITTHSFNCGGEFFYCNTSGLFNSTWIGNGTKNNNNTNDTITLPCRIKQIINMWQRVGQPMYAPPIQGKIRCVSNITGLLLTRDGGNNNTNETETFRPGGGDMRDNWRSELYKYKVVKIEPLGVAPTRCKRRVVEGGGGSGGGGSAVGIGAVFLGFLGAAGSTMGAASMTLTVQARNLLSGGSGSGSGSTVWGIKQLQARVLAVERYLRDQQLLGIWGCSGKLICCTNVPWNSSWSNKSQDEIWDNMTWMEWDKEINNYTDIIYSLIEESQNQQEKNEQDLLALDKWASLWNWFDITNWLWYIKAIIVAALVLSILSIIISLLFCCWAYVATKEIRRINFKTNHINTISSSVDDLIRYRRRRRRGSGEGRGSLLTCGDVEENPGPMGCVQCKDKEAGSQIKIPLPKPPDSDSQRLNAFPVIMAQEGKGRLLRQIRLRKILSGDPSDQQITFVNTYGFIRATPETSEFISESSQQKVTPVVTACMLSFGAGPVLEDPQHMLKALDQTDIRVRKTASDKEQILFEINRIPNLFRHHQISADHLIQASSDKYVKSPAKLIAGVNYIYCVTFLSVTVCSASLKFRVARPLLAARSRLVRAVQMEVLLRVTCKKDSQMAKSMLNDPDGEGCIASVWFHLCNLCKGRNKLRSYDENYFASKCRKMNLTVSIGDMWGPTILVHAGGHIPTTAKPFFNSRGWVCHPIHQSSPSLAKTLWSSGCEIKAASAILQGSDYASLAKTDDIIYSKIKVDKDAANYKGVSWSPFRKSASMSNL

SEQ ID NO: 23 - DNA, 3375 bp, Artificial Sequence
  Fusion protein nucleotide sequence
atggacagagccaaactgctgctgctcctgttgctcctcctgctgcctcaggctcaggccgtggaaaatctgtgggtcaccgtgtactacggcgtgcccgtgtggaaggatgccgagacaacactgttctgtgccagcgacgccaaggcctacgataccgaagtgcggaatgtgtgggccactcacgcctgcgttcccaccgatcctaatcctcaagagatcgtgctggaaaacgtgaccgagaacttcaacatgtggaagaacaacatggtcgagcagatgcacaccgacatcatcagcctgtgggaccagagcctgaagccttgcgtgaagctgacccctctgtgcgtgaccctgaactgcaccaacgtgaacgtgaccaacaccaccaacaacaccgaggaaaagggcgagatcaagaactgcagcttcaacatcaccaccgagctgcgggacaagaaaaagaaggtgtacgccctgttctaccggctggacgtggtgcccatcgacgataacaacaacaactccagcaattaccggctgatcaactgcaacaccagcgccatcactcaggcctgtcctaaggtgtccttcgagcccattcctatccactactgtgcccctgccggcttcgccatcctgaagtgcaacgacaagaagttcaacggcacaggcccctgcaagaacgtgtccaccgtgcagtgtacccacggcatcaagccagtggtgtctacccagctgctgctgaatggctctctggccgaggaagagatcatcatcagaagcgagaacatcacgaacaacgccaagaccatcatcgtgcagctgaacgagagcgtggaaatcaattgcacccggcctaacaacaatacccggaagtccatcagaatcggccctggccagtggttttatgccaccggcgatattatcggcgacatcagacaggcccactgtaacatcagcggcaccaagtggaacaagaccctgcagcaggtcgtgaagaagctgagagagcacttcaacaacaagacgatcatcttcaaccccagctctggcggcgacctggaaatcaccacacacagcttcaattgtggcggcgagttcttctactgcaatacctccggcctgttcaacagcacctggatcggcaatggcaccaagaacaacaacaacaccaacgacaccatcacactgccctgccggatcaagcagatcatcaatatgtggcagcgcgtgggccagcctatgtacgctcctccaatccagggcaagatcagatgcgtgtccaatatcaccggcctgctgctcacaagagatggcggaaacaacaacacgaatgagacagagacattcagacccggcggaggcgacatgagagacaattggagaagcgagctgtacaagtacaaggtggtcaagatcgagcccctgggcgtcgcacctacacggtgcaaaagaagagtggtcgaaggcggcggaggaagcggaggcggaggatctgctgttggaatcggagccgtgttcctgggctttctgggagccgctggatctacaatgggcgctgccagcatgaccctgacagtgcaggctagaaatctgctgtctggcggcagcggctctggctcaggatctacagtgtggggaatcaagcagctgcaggccagagtgctggccgtcgaaagatacctgagagatcagcagctcctcggcatctggggctgttctggcaagctgatctgctgcaccaatgtgccctggaacagctcctggtccaacaagagccaggacgagatctgggacaacatgacctggatggaatgggacaaagagattaacaactatacggacatcatctacagcctgatcgaggaaagccagaaccagcaagagaagaacgagcaggacctgctggccctggataagtgggctagcctgtggaattggttcgacatcaccaactggctgtggtacatcaaggccatcattgtggccgctctggtgctgagcatcctgtccatcatcatctccctgctgttctgctgctgggcctacgtggccaccaaagagatcagacggatcaacttcaagaccaaccacatcaacaccatcagctccagcgtggacgacctgatcagataccggcggagaagaagaagaggctccggcgaaggcagaggcagccttcttacatgtggcgacgtggaagagaaccccggacctatgggatgcgtgcagtgcaaagacaaagaggccggcagccagatcaagatccctctgcctaagcctcctgacagcgacagccagagactgaacgctttccccgtgatcatggcccaagaaggcaagggcagactgctgcggcagatccggctgagaaagatcctcagcggcgaccctagcgaccagcagattaccttcgtgaacacctacggcttcatccgggccacacctgagacaagcgagttcatcagcgagagcagccagcagaaagtgacccctgtggtcaccgcctgcatgctgtcttttggagccggacctgtgctggaagatccccagcacatgctgaaagccctggaccagacagacatcagagtgcgcaagaccgccagcgacaaagagcagattctgttcgagatcaacaggattcccaacctgttccggcaccaccagatcagcgccgatcatctgattcaggccagctccgacaaatacgtgaagtcccctgccaagctgattgccggcgtgaactatatctactgcgtgaccttcctgagcgtgaccgtgtgtagcgcctctctgaagtttagagtggccagacctctgctggccgccagatccagacttgttagagccgtgcagatggaagtgctgctgagagtgacctgcaaaaaggactcccagatggccaagagcatgctgaacgaccctgatggcgagggctgtatcgccagcgtgtggttccacctgtgcaatctgtgcaaaggccggaacaagctgcggagctacgacgagaattacttcgccagcaagtgccggaagatgaacctgaccgtgtccatcggcgatatgtggggccctacaatcctggtgcatgccggcggacacatccctacaaccgccaagccattcttcaactccagaggctgggtctgccatccaatccaccagtctagtcccagcctggccaagacactgtggtctagcggctgcgaaatcaaagccgccagcgctatcctgcagggctctgattatgcctctctggctaagaccgacgacattatctactccaagatcaaggtggacaaggacgccgccaactacaagggagtcagctggtccccattcagaaagtccgccagcatgtccaacctgtag

SEQ ID NO: 24 - protein, 1117 aa, Artificial Sequence
  Fusion protein - PIV5 matrix protein, a MTS derived from a Fyn-like protein kinase, a PIV5 TMD and CT fused to HIV antigen and a T2A sequence
MDRAKLLLLLLLLLLPQAQAVENLWVTVYYGVPVWKDAETTLFCASDAKAYDTEVRNVWATHACVPTDPNPQEIVLENVTENFNMWKNNMVEQMHTDIISLWDQSLKPCVKLTPLCVTLNCTNVNVTNTTNNTEEKGEIKNCSFNITTELRDKKKKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISGTKWNKTLQQVVKKLREHFNNKTIIFNPSSGGDLEITTHSFNCGGEFFYCNTSGLFNSTWIGNGTKNNNNTNDTITLPCRIKQIINMWQRVGQPMYAPPIQGKIRCVSNITGLLLTRDGGNNNTNETETFRPGGGDMRDNWRSELYKYKVVKIEPLGVAPTRCKRRVVEGGGGSGGGGSAVGIGAVFLGFLGAAGSTMGAASMTLTVQARNLLSGGSGSGSGSTVWGIKQLQARVLAVERYLRDQQLLGIWGCSGKLICCTNVPWNSSWSNKSQDEIWDNMTWMEWDKEINNYTDIIYSLIEESQNQQEKNEQDLLALDKWASLWNWFDITNWLWYIKVLSIIAICLGSLGLILIILLSVVVWKLLTIVVANRNRMENFVYHKRRRRRRGSGEGRGSLLTCGDVEENPGPMGCVQCKDKEPSISIPADPTNPRQSIKAFPIVINSDGGEKGRLVKQLRTTYLNDLDTHEPLVTFVNTYGFIYEQDRGNTIVGEDQLGKKREAVTAAMVTLGCGPNLPSLGNVLGQLSEFQVIVRKTSSKAEEMVFEIVKYPRIFRGHTLIQKGLVCVSAEKFVKSPGKVQSGMDYLFIPTFLSVTYCPAAIKFQVPGPMLKMRSRYTQSLQLELMIRILCKPDSPLMKVHIPDKEGRGCLVSVWLHVCNIFKSGNKNGSEWQEYWMRKCANMQLEVSIADMWGPTIIIHARGHIPKSAKLFFGKGGWSCHPLHEVVPSVTKTLWSVGCEITKAKAIIQESSISLLVETTDIISPKVKISSKHRRFGKSNWGLFKKTKSLPNLTELE

SEQ ID NO: 25 - DNA, 3354 bp, Artificial Sequence; identical to SEQ ID NO: 8
  Fusion protein nucleotide sequence

SEQ ID NO: 26 - protein, 6 aa, Unknown
  Furin sequence
  misc_feature (2)..(2): Xaa can be any naturally occurring amino acid
  misc_feature (5)..(5): Xaa can be any naturally occurring amino acid
RXRKXR

SEQ ID NO: 27 - protein, 6 aa, Unknown
  Optimised furin sequence
RRRRRR

SEQ ID NO: 28 - protein, 19 aa, Unknown
  Porcine teschovirus-1 2A
ATNFSLLKQAGDVEENPGP

SEQ ID NO: 29 - protein, 20 aa, Unknown
  Thosea asigna virus 2A
QCTNYALLKLAGDVESNPGP

SEQ ID NO: 30 - protein, 22 aa, Unknown
  Equine rhinitis A virus 2A and Foot and mouth disease virus 2A
VKQTLNFDLLKLAGDVESNPGP

SEQ ID NO: 31 - DNA, 72 bp, Unknown
  MuV TMD nucleotide sequence
gtgctgagcatcattgccatctgcctgggcagcctgggcctgatcctgatcattctgctgagcgtggtcgtg

SEQ ID NO: 32 - DNA, 63 bp, Unknown
  MuV CT nucleotide sequence
tggaaactgctgacaatcgtggtggccaaccggaaccggatggaaaacttcgtgtaccacaag

SEQ ID NO: 33 - DNA, 78 bp, Unknown
  PIV5 TMD nucleotide sequence
gccatcattgtggccgctctggtgctgagcatcctgtccatcatcatctccctgctgttctgctgctgggcctacgtg

SEQ ID NO: 34 - DNA, 84 bp, Unknown
  PIV5 CT nucleotide sequence
gccaccaaagagatcagacggatcaacttcaagaccaaccacatcaacaccatcagctccagcgtggacgacctgatcagatac

SEQ ID NO: 35 - protein, 4 aa, Unknown
  Furin sequence
  misc_feature (2)..(3): Xaa can be any naturally occurring amino acid
RXXR

SEQ ID NO: 36 - protein, 4 aa, Unknown
  Furin sequence
  misc_feature (2)..(2): Xaa can be any naturally occurring amino acid
  MISC_FEATURE (3)..(3): X = arginine or lysine
RXXR

SEQ ID NO: 37 - protein, 4 aa, Unknown
  Furin sequence
RRRR

(19) United States
(12) Patent Application Publication
(10) Pub. No.: US 2021/0386850 A1
(43) Pub. Date: Dec. 16, 2021

(54) FUSION PROTEIN

(71) Applicant: Imperial College Innovations Limited, London (GB)

(72) Inventors: Robin Shattock, London (GB); Yoann Aldon, London (GB); Paul McKay, London (GB)

(21) Appl. No.: 17/285,786

(22) PCT Filed: Oct. 16, 2019
(86) PCT No.: PCT/GB2019/052945

§ 371 Date: Apr. 15, 2021

(30) Foreign Application Priority Data
Oct. 17, 2018 (GB) 1816873.2

(51) Int. Cl. A61K 39/155; C12N 7/00; C07K 14/11; C07K 14/115

(52) U.S. Cl. CPC A61K 39/155; C12N 7/00; C07K 14/11; C07K 14/115; C12N 2760/16023; A61K 2039/5258; C12N 2760/16071; C12N 2760/18023; C12N 2760/18034; C12N 2760/18071; C12N 2760/16034

(57) ABSTRACT

The invention relates to fusion proteins, and to the use of fusion proteins (or genetic constructs or vectors encoding such fusion proteins) to vaccinate against viral infections. The invention extends to pharmaceutical compositions comprising such fusion proteins or constructs for preventing and treating viral infections, and to methods and uses thereof.

The present invention relates to fusion proteins, and in particular to the use of fusion proteins (or genetic constructs or vectors encoding such fusion proteins) to vaccinate against viral infections. The invention extends to pharmaceutical compositions comprising such fusion proteins or constructs for preventing and treating viral infections, and to methods and uses thereof.

Presenting an antigen in the appropriate context to preserve or mimic its native antigenicity is likely necessary to induce relevant systemic and mucosal immune responses. To date, the majority of commercialized vaccines generate protection against pathogens through the induction of an efficient humoral immune response (1). Live attenuated viruses or killed viruses can be used for vaccine purposes (2). However, for some viruses, such as the human immunodeficiency virus (HW) and the Ebola virus, these classical approaches are excluded for safety reasons (3). As the main target to develop a sterilizing immunity against such viruses is their surface glycoprotein(s) (GP), which mediate attachment and fusion to the target cells (4-6), efforts to develop subunit vaccines using soluble recombinant GPs as antigens have driven considerable efforts into stabilizing these proteins to closely mimic the native extracellular domain of the protein presented on virion surfaces (7, 8). While these efforts have led to the production of well-characterized immunogens, these proteins are not presented in the context of a viral membrane. Therefore, another approach for the presentation of viral GP antigens in an appropriate context is the use of a particular type of subunit vaccine: virus-like particles (VLP) (9-11).

VLPs are able to reproduce the structure of a virus and have proven successful in humans for several non-enveloped vaccines, such as human papilloma virus (HPV), hepatitis B virus (HBV) and hepatitis E virus (HEV) (9). VLPs are recognised by the immune system similarly to viruses and present the viral immunogens in a more relevant conformation than soluble recombinant proteins. One major advantage of the VLP approach is its versatility, as multiple antigens from the same or from different pathogens can be co-expressed to build VLPs with different characteristics (12, 13). This versatility also allows the design of VLPs that can overcome issues of specific antigens.

However, for viruses such as HIV, HIV virions present a restricted number of Envelope (Env) GPs on their surface (<20), which does not provide sufficient valency to trigger potent B cell receptor (BCR) engagement for antibody responses (14). Thus, the use of VLPs to vaccinate against such viruses is limited.

There is therefore a need to provide alternative approaches to vaccinate against viral infections, for example those which produce lower numbers of Envelope GPs, such as HIV.

The inventors have developed a novel platform approach that utilises modified Paramyxovirus or Orthomyxovirus matrix proteins to generate VLPs that can be subsequently pseudotyped (i.e. decorated) with any viral glycoprotein demonstrating broad applicability. However, in addition to this platform technology, the inventors have also developed a novel fusion protein that enables the highly efficient display of viral antigens (e.g. on a VLP) that would otherwise not provide sufficient valency to trigger potent B cell receptor (BCR) engagement.

Accordingly, in a first aspect of the invention, there is provided a fusion protein comprising an antigen, and a Paramyxovirus or Orthomyxovirus transmembrane domain (TMD) and/or a Paramyxovirus or Orthomyxovirus cytoplasmic tail (CT).

In one embodiment, the antigen may be non-viral, for example antigens associated with pathogenic protozoa, such as Plasmodium falciparum, Plasmodium malariae, Plasmodium ovale, and Plasmodium vivax (e.g. the C-terminus and central repeat region of the malaria circumsporozoite protein). Additional immunogens non-viral antigens may be associated with Toxoplasma gondii; Trypanosoma brucei, Trypanosoma cruzi; Schistosoma haematobium; Schistosoma mansoni; Schistosoma japonicum; Leishmania donovani; Giardia intestinalis; or Cryptosporidium parvum. These antigens may be useful for vaccinating against infection with any of these protozoa.

In another embodiment, the non-viral antigen may a bacterial immunogen, such as immunogens associated with (e.g., synthesized by and endogenous to) any pathogenic bacteria, including, e.g., pathogenic gram positive bacteria, such as pathogenic Pasteurella species, Staphylococci species, and Streptococcus species; and gram-negative pathogens, such as those of the genera Neisseria, Escherichia, Bordetella, Campylobacter, Legionella, Pseudomonas, Shigella, Vibrio, Yersinia, Salmonella, Haemophilus, Brucella, Francisella, Bacterioides, Helicobacter pyloris, Borelia burgdorferi, Legionella pneumophila, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Chlamydia trachomatis, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis or Treponema pallidium. These antigens may be useful for vaccinating against infection with any of these bacteria.

In another embodiment, the antigen may be a tumour-associated antigen, wherein the tumour associated antigen is fused to the transmembrane domain of an Orthomyxovirus or Paramyxovirus. The whole or part of the tumour-associated antigen may be present in the fusion protein. Typical tumour antigens include antigens from: breast cancer (e.g. HER-2 antigen); pancreatic cancer (e.g. Trop2, hMSLN), prostate cancer (PSA), Skin cancer (e.g. MAGE-3, MAA), lung cancer (e.g. CLDN18.2), ovarian cancer (OV-TL3 and MOV18), renal tumour-associated antigen (e.g. G250, EGP-40). These antigens may be useful for vaccinating against any of these cancers.

In another embodiment, the fusion protein may comprise a peptide antigen associated with neurodegenerative diseases (for example. a protein associated with Alzheimer's disease, e.g. beta amyloid or tau protein), autoimmune diseases (for example proteins associated with arthritis, e.g. TNF-alpha, IL-1 alpha), allergy (e.g. Der p I or Der f I). These antigens may be useful for vaccinating against any of these diseases.

However, in a preferred embodiment, the antigen is a viral antigen, and the TMD and/or CT is derived from a different virus from that of the viral antigen. Preferably, the viral antigen is derived from an envelope virus selected from the group consisting: Retroviridae (e.g. HIV-1, HIV-2); Togaviridae (e.g Rubella virus, alphavirus); Arenaviridae (e.g. e.g. Lassa virus, Lymphocytic choriomeningitis virus); Flaviviridae (e.g. Dengue virus, hepatitis C virus, yellow fever virus, Zika virus); Orthomyxoviridae (e.g. influenza virus A, influenza virus B, influenza virus C, isavirus, thogotovirus);

Paramyxoviridae (e.g. measles virus, mumps virus, respiratory syncytial virus, Rinderpest virus, canine distemper virus, Nipha virus); Bunyaviridae (e.g. California encephalitis virus, hantavirus); Rhabdoviridae (eg. Rabies virus); Filoviridae (e.g. Ebola virus, Marburg virus); Coronaviridae (e.g. Corona virus, SARS); Bornaviridae (e.g. Borna disease virus); and Arteriviridae (e.g. Arterivirus, equine arteritis virus). More preferably, the viral antigen is derived from HIV. These antigens may be useful for vaccinating against infection with any of these viruses.

Preferably, the viral antigen is a viral envelope protein, and more preferably a viral glycoprotein. The viral antigen is preferably a class I trimeric viral glycoprotein, as expressed by the following viral families: Retroviridae (e.g. HIV-1, HIV-2); Filoviridae (e.g. Ebola virus, Marburg virus); Orthomyxoviridae (e.g. influenza virus A, influenza virus B, influenza virus C, isavirus, thogotovirus); Paramyxoviridae (e.g. measles virus, mumps virus, respiratory syncytial virus, Rinderpest virus, canine distemper virus, Nipha virus); Coronaviridae (e.g. Corona virus, SARS), a class III trimeric protein, as expressed by the following: vesicular stomatitis virus (VSV), herpes simplex virus 1 (HSV-1 and 2) and Epstein-Barr Virus (EBV) gB, and class II glycoproteins of Flaviviridae (e.g. Dengue virus, hepatitis C virus, yellow fever virus, Zika virus); Bunyaviridae (e.g. Rift Valley fever); Togaviridae (e.g. Rubella virus, alphavirus). Preferably, the viral antigen is a class 1 trimeric viral glycoprotein expressed by Retroviridae (e.g. HIV-1, HIV-2).

In one embodiment, the TMD or CT comprises a Paramyxovirus TMD or CT. The Paramyxovirus may be selected from the group consisting of: Rubulavirus (Mumps); Parainfluenza virus 5 (also known as Simian virus 5); Parainfluenza virus 2; Parainfluenza virus 3; Respirovirus (for example, Sendai virus); Morbillivirus (for example, Measles virus); Henipavirus (for example, Nipah virus); Avulavirus (for example, Newcastle disease virus); Pneumovirus (for example, Human respiratory syncytial virus); and Metapneumovirus (for example, Human metapneumovirus).

In one embodiment, the TMD or CT comprises an Orthomyxovirus TMD or CT. The Orthomyxovirus may be selected from the group consisting of: influenza virus A; influenza virus B; and influenza virus C.

Preferably, the fusion protein comprises a viral antigen and a Paramyxovirus TMD and/or a Paramyxovirus CT. Most preferably, however, the fusion protein comprises a viral antigen and a Paramyxovirus TMD and a Paramyxovirus CT.

More preferably, the fusion protein comprises a Mumps virus (MuV) or Parainfluenzavirus 5 (PIV5) TMD and/or CT. In one preferred embodiment, the fusion protein comprises a viral antigen and a Mumps virus (MuV) TMD and MuV CT. In another preferred embodiment, the fusion protein comprises a viral antigen and a Parainfluenzavirus 5 (PIV5) TMD and PIV5 CT.

The fusion protein is preferably configured to display the antigen, preferably a virus antigen, on a virus-like particle (VLP). Preferably, the term VLP described in all aspects of the invention relates to an enveloped VLP, i.e. one which is enveloped by a membrane envelope.

The skilled person would understand that antigen display relates to the display of viral proteins (i.e. antigens), which when engaged by a B cell receptor (BCR), activate the B cells and leads to the production of specific antibodies to the viral protein or antigen. The skilled person will appreciate that antigen display encompasses the term “pseudotyping”, which relates to the display or decoration of viral proteins (most preferably, glycoproteins) on the outer surface of generated VLPs.

The TMD and/or CT may be disposed N-terminal to the antigen in the fusion protein. However, the TMD and/or CT is preferably disposed C-terminal to the antigen in the fusion protein. In an embodiment in which the fusion protein comprises a TMD and a CT, then the CT may be N-terminal to the TMD. However, in a preferred embodiment, the CT is C-terminal to the TMD.

In one embodiment, the amino acid sequence of Mumps virus (MuV) TMD is provided herein as SEQ ID NO:1, as follows:

[SEQ ID No: 1]
VLSIIAICLGSLGLILIILLSVVV

Hence, preferably the fusion protein comprises an amino acid sequence substantially as set out in SEQ ID NO: 1, or a biologically active variant or fragment thereof.

In one embodiment, the amino acid sequence of Mumps virus (MuV) CT is provided herein as SEQ ID NO:2, as follows:

[SEQ ID No: 2]
WKLLTIVVANRNRMENFVYHK

Hence, preferably the fusion protein comprises an amino acid sequence substantially as set out in SEQ ID NO:2, or a biologically active variant or fragment thereof.

In one embodiment, the amino acid sequence of PIV5 TMD is provided herein as SEQ ID NO:3, as follows:

[SEQ ID No: 3]
AIIVAALVLSILSIIISLLFCCWAYV

Hence, preferably the fusion protein comprises an amino acid sequence substantially as set out in SEQ ID NO: 3, or a biologically active variant or fragment thereof.

In one embodiment, the amino acid sequence of PIV5 CT is provided herein as SEQ ID NO:4, as follows:

[SEQ ID No: 4]
ATKEIRRINFKTNHINTISSSVDDLIRY

Hence, preferably the fusion protein comprises an amino acid sequence substantially as set out in SEQ ID NO: 4, or a biologically active variant or fragment thereof.

As shown below, the fusion protein may comprise a MuV TMD and CT (underlined) fused to a HIV antigen (e.g. HIV-1 Env). Thus, in one embodiment, the amino acid sequence of the fusion protein is provided herein as SEQ ID NO: 5, as follows:

[SEQ ID No: 5]
MDRAKLLLLLLLLLLPQAQAVENLWVTVYYGVPVWKDAETTLFCASDAKA
YDTEVRNVWATHACVPTDPNPQEIVLENVTENFNMWKNNMVEQMHTDIIS
LWDQSLKPCVKLTPLCVTLNCTNVNVTNTTNNTEEKGEIKNCSFNITTEL
RDKKKKVYALFYRLDVVPIDDNNNNSSNYRLINCNTSAITQACPKVSFEP
IPIHYCAPAGFAILKCNDKKFNGTGPCKNVSTVQCTHGIKPVVSTQLLLN
GSLAEEEIIIRSENITNNAKTIIVQLNESVEINCTRPNNNTRKSIRIGPG
QWFYATGDIIGDIRQAHCNISGTKWNKTLQQVVKKLREHFNNKTIIFNPS
SGGDLEITTHSFNCGGEFFYCNTSGLFNSTWIGNGTKNNNNTNDTITLPC
RIKQIINMWQRVGQPMYAPPIQGKIRCVSNITGLLLTRDGGNNNTNETET
FRPGGGDMRDNWRSELYKYKVVKIEPLGVAPTRCKRRVVEGGGGSGGGGS
AVGIGAVFLGFLGAAGSTMGAASMTLTVQARNLLSGGSGSGSGSTVWGIK
QLQARVLAVERYLRDQQLLGIWGCSGKLICCTNVPWNSSWSNKSQDEIWD
NMTWMEWDKEINNYTDIIYSLIEESQNQQEKNEQDLLALDKWASLWNWFD
ITNWLWYIKAIIVAALVLSILSIIISLLFCCWAYVATKEIRRINFKTNHI
NTISSSVDDLIRY

Hence, preferably the fusion protein comprises an amino acid sequence substantially as set out in SEQ ID NO: 5, or a biologically active variant or fragment thereof.

In one embodiment, the fusion protein may be encoded by a nucleic acid having a nucleotide sequence, which is provided herein as SEQ ID NO:6, as follows:

[SEQ ID No: 6]
ATGGACAGAGCCAAACTGCTGCTGCTCCTGTTGCTCCTCCTGCTGCCTCA
GGCTCAGGCCGTGGAAAATCTGTGGGTCACCGTGTACTACGGCGTGCCCG
TGTGGAAGGATGCCGAGACAACACTGTTCTGTGCCAGCGACGCCAAGGCC
TACGATACCGAAGTGCGGAATGTGTGGGCCACTCACGCCTGCGTTCCCAC
CGATCCTAATCCTCAAGAGATCGTGCTGGAAAACGTGACCGAGAACTTCA
ACATGTGGAAGAACAACATGGTCGAGCAGATGCACACCGACATCATCAGC
CTGTGGGACCAGAGCCTGAAGCCTTGCGTGAAGCTGACCCCTCTGTGCGT
GACCCTGAACTGCACCAACGTGAACGTGACCAACACCACCAACAACACCG
AGGAAAAGGGCGAGATCAAGAACTGCAGCTTCAACATCACCACCGAGCTG
CGGGACAAGAAAAAGAAGGTGTACGCCCTGTTCTACCGGCTGGACGTGGT
GCCCATCGACGATAACAACAACAACTCCAGCAATTACCGGCTGATCAACT
GCAACACCAGCGCCATCACTCAGGCCTGTCCTAAGGTGTCCTTCGAGCCC
ATTCCTATCCACTACTGTGCCCCTGCCGGCTTCGCCATCCTGAAGTGCAA
CGACAAGAAGTTCAACGGCACAGGCCCCTGCAAGAACGTGTCCACCGTGC
AGTGTACCCACGGCATCAAGCCAGTGGTGTCTACCCAGCTGCTGCTGAAT
GGCTCTCTGGCCGAGGAAGAGATCATCATCAGAAGCGAGAACATCACGAA
CAACGCCAAGACCATCATCGTGCAGCTGAACGAGAGCGTGGAAATCAATT
GCACCCGGCCTAACAACAATACCCGGAAGTCCATCAGAATCGGCCCTGGC
CAGTGGTTTTACGCCACCGGCGATATCATCGGCGACATCAGACAGGCCCA
CTGTAACATCAGCGGCACCAAGTGGAACAAGACCCTGCAGCAGGTCGTGA
AGAAGCTGAGAGAGCACTTCAACAACAAGACGATCATCTTCAACCCCAGC
TCTGGCGGCGACCTGGAAATCACCACACACAGCTTCAATTGTGGCGGCGA
GTTCTTCTACTGCAATACCTCCGGCCTGTTCAACAGCACCTGGATCGGCA
ATGGCACCAAGAACAACAACAACACCAACGACACCATCACACTGCCCTGC
CGGATCAAGCAGATCATCAATATGTGGCAGCGCGTGGGCCAGCCTATGTA
CGCTCCTCCAATCCAGGGCAAGATCAGATGCGTGTCCAATATCACCGGCC
TGCTGCTCACAAGAGATGGCGGAAACAACAACACGAATGAGACAGAGACA
TTCAGACCCGGCGGAGGCGACATGAGAGACAATTGGAGAAGCGAGCTGTA
CAAGTACAAGGTGGTCAAGATCGAGCCCCTGGGCGTCGCACCTACACGGT
GCAAAAGAAGAGTGGTCGAAGGCGGCGGAGGAAGCGGAGGCGGAGGATCT
GCTGTTGGAATCGGAGCCGTGTTCCTGGGCTTTCTGGGAGCCGCTGGATC
TACAATGGGCGCTGCCAGCATGACCCTGACAGTGCAGGCTAGAAATCTGC
TGTCTGGCGGCAGCGGCTCTGGCTCAGGATCTACAGTGTGGGGAATCAAG
CAGCTGCAGGCCAGAGTGCTGGCCGTCGAGAGATACCTGAGAGATCAGCA
GCTCCTCGGCATCTGGGGCTGTTCTGGCAAGCTGATCTGCTGCACCAATG
TGCCCTGGAACAGCTCCTGGTCCAACAAGAGCCAGGACGAGATCTGGGAC
AACATGACCTGGATGGAATGGGACAAAGAGATTAACAACTACACGGATAT
CATCTACAGCCTGATCGAGGAAAGCCAGAACCAGCAAGAGAAGAACGAGC
AGGACCTGCTGGCCCTGGATAAGTGGGCTTCCCTGTGGAATTGGTTCGAC
ATCACCAACTGGCTGTGGTACATCAAGGCCATCATTGTGGCCGCTCTGGT
GCTGAGCATCCTGTCCATCATCATCTCCCTGCTGTTCTGCTGCTGGGCCT
ACGTGGCCACCAAAGAGATCAGACGGATCAACTTCAAGACCAACCACATC
AACACCATCAGCTCCAGCGTGGACGACCTGATCCGGTACTAG

Hence, preferably the fusion protein is encoded by a nucleic acid comprising a nucleotide sequence substantially as set out in SEQ ID NO: 6, or a variant or fragment thereof.

The fusion protein may comprise a PIV5 TMD and CT (underlined) fused to HIV antigen (e.g. HIV-1 Env). Thus, in another embodiment, the fusion protein is provided herein as SEQ ID NO: 7, as follows:

[SEQ ID No: 7]
MDRAKLLLLLLLLLLPQAQAVENLWVTVYYGVPVWKDAETTLFCASDAKA
YDTEVRNVWATHACVPTDPNPQEIVLENVTENFNMWKNNMVEQMHTDIIS
LWDQSLKPCVKLTPLCVTLNCTNVNVTNTTNNTEEKGEIKNCSFNITTEL
RDKKKKVYALFYRLDVVPIDDNNNNSSNYRLINCNTSAITQACPKVSFEP
IPIHYCAPAGFAILKCNDKKFNGTGPCKNVSTVQCTHGIKPVVSTQLLLN
GSLAEEEIIIRSENITNNAKTIIVQLNESVEINCTRPNNNTRKSIRIGPG
QWFYATGDIIGDIRQAHCNISGTKWNKTLQQVVKKLREHFNNKTIIFNPS
SGGDLEITTHSFNCGGEFFYCNTSGLFNSTWIGNGTKNNNNTNDTITLPC
RIKQIINMWQRVGQPMYAPPIQGKIRCVSNITGLLLTRDGGNNNTNETET
FRPGGGDMRDNWRSELYKYKVVKIEPLGVAPTRCKRRVVEGGGGSGGGGS
AVGIGAVFLGFLGAAGSTMGAASMTLTVQARNLLSGGSGSGSGSTVWGIK
QLQARVLAVERYLRDQQLLGIWGCSGKLICCTNVPWNSSWSNKSQDEIWD
NMTWMEWDKEINNYTDIIYSLIEESQNQQEKNEQDLLALDKWASLWNWFD
ITNWLWYIKVLSIIAICLGSLGLILIILLSVVVWKLLTIVVANRNRMENF
VYHK

Hence, preferably the fusion protein comprises an amino acid sequence substantially as set out in SEQ ID NO: 7, or a biologically active variant or fragment thereof.

In one embodiment, the fusion protein may be encoded by a nucleic acid having a nucleotide sequence, which is provided herein as SEQ ID NO: 8, as follows:

[SEQ ID No: 8]
ATGGACAGAGCCAAACTGCTGCTGCTCCTGTTGCTCCTCCTGCTGCCTCA
GGCTCAGGCCGTGGAAAATCTGTGGGTCACCGTGTACTACGGCGTGCCCG
TGTGGAAGGATGCCGAGACAACACTGTTCTGTGCCAGCGACGCCAAGGCC
TACGATACCGAAGTGCGGAATGTGTGGGCCACTCACGCCTGCGTTCCCAC
CGATCCTAATCCTCAAGAGATCGTGCTGGAAAACGTGACCGAGAACTTCA
ACATGTGGAAGAACAACATGGTCGAGCAGATGCACACCGACATCATCAGC
CTGTGGGACCAGAGCCTGAAGCCTTGCGTGAAGCTGACCCCTCTGTGCGT
GACCCTGAACTGCACCAACGTGAACGTGACCAACACCACCAACAACACCG
AGGAAAAGGGCGAGATCAAGAACTGCAGCTTCAACATCACCACCGAGCTG
CGGGACAAGAAAAAGAAGGTGTACGCCCTGTTCTACCGGCTGGACGTGGT
GCCCATCGACGATAACAACAACAACTCCAGCAATTACCGGCTGATCAACT
GCAACACCAGCGCCATCACTCAGGCCTGTCCTAAGGTGTCCTTCGAGCCC
ATTCCTATCCACTACTGTGCCCCTGCCGGCTTCGCCATCCTGAAGTGCAA
CGACAAGAAGTTCAACGGCACAGGCCCCTGCAAGAACGTGTCCACCGTGC
AGTGTACCCACGGCATCAAGCCAGTGGTGTCTACCCAGCTGCTGCTGAAT
GGCTCTCTGGCCGAGGAAGAGATCATCATCAGAAGCGAGAACATCACGAA
CAACGCCAAGACCATCATCGTGCAGCTGAACGAGAGCGTGGAAATCAATT
GCACCCGGCCTAACAACAATACCCGGAAGTCCATCAGAATCGGCCCTGGC
CAGTGGTTTTATGCCACCGGCGATATTATCGGCGACATCAGACAGGCCCA
CTGTAACATCAGCGGCACCAAGTGGAACAAGACCCTGCAGCAGGTCGTGA
AGAAGCTGAGAGAGCACTTCAACAACAAGACGATCATCTTCAACCCCAGC
TCTGGCGGCGACCTGGAAATCACCACACACAGCTTCAATTGTGGCGGCGA
GTTCTTCTACTGCAATACCTCCGGCCTGTTCAACAGCACCTGGATCGGCA
ATGGCACCAAGAACAACAACAACACCAACGACACCATCACACTGCCCTGC
CGGATCAAGCAGATCATCAATATGTGGCAGCGCGTGGGCCAGCCTATGTA
CGCTCCTCCAATCCAGGGCAAGATCAGATGCGTGTCCAATATCACCGGCC
TGCTGCTCACAAGAGATGGCGGAAACAACAACACGAATGAGACAGAGACA
TTCAGACCCGGCGGAGGCGACATGAGAGACAATTGGAGAAGCGAGCTGTA
CAAGTACAAGGTGGTCAAGATCGAGCCCCTGGGCGTCGCACCTACACGGT
GCAAAAGAAGAGTGGTCGAAGGCGGCGGAGGAAGCGGAGGCGGAGGATCT
GCTGTTGGAATCGGAGCCGTGTTCCTGGGCTTTCTGGGAGCCGCTGGATC
TACAATGGGCGCTGCCAGCATGACCCTGACAGTGCAGGCTAGAAATCTGC
TGTCTGGCGGCAGCGGCTCTGGCTCAGGATCTACAGTGTGGGGAATCAAG
CAGCTGCAGGCCAGAGTGCTGGCCGTCGAAAGATACCTGAGAGATCAGCA
GCTCCTCGGCATCTGGGGCTGTTCTGGCAAGCTGATCTGCTGCACCAATG
TGCCCTGGAACAGCTCCTGGTCCAACAAGAGCCAGGACGAGATCTGGGAC
AACATGACCTGGATGGAATGGGACAAAGAGATTAACAACTATACGGACAT
CATCTACAGCCTGATCGAGGAAAGCCAGAACCAGCAAGAGAAGAACGAGC
AGGACCTGCTGGCCCTGGATAAGTGGGCTTCCCTGTGGAATTGGTTCGAC
ATCACCAACTGGCTGTGGTACATCAAGGTGCTGAGCATCATTGCCATCTG
CCTGGGCAGCCTGGGCCTGATCCTGATCATTCTGCTGAGCGTGGTCGTGT
GGAAACTGCTGACAATCGTGGTGGCCAACCGGAACCGGATGGAAAACTTC
GTGTACCACAAGCGGCGCAGAAGGCGGAGAGGATCTGGCGAAGGCAGAGG
CTCTCTGCTGACATGTGGCGACGTGGAAGAGAACCCTGGACCTATGGGAT
GCGTGCAGTGCAAGGACAAAGAACCCAGCATCAGCATCCCCGCCGATCCT
ACAAACCCCAGACAGAGCATCAAGGCCTTTCCAATCGTGATCAACAGCGA
CGGCGGCGAGAAGGGCAGACTGGTTAAGCAGCTGAGAACCACCTACCTGA
ACGACCTGGACACCCACGAGCCTCTGGTCACCTTCGTGAACACCTACGGC
TTCATCTACGAACAGGACCGGGGCAACACAATCGTCGGCGAAGATCAGCT
GGGCAAGAAACGGGAAGCCGTGACAGCCGCCATGGTCACACTTGGCTGTG
GCCCTAATCTGCCTAGCCTGGGCAATGTGCTTGGCCAGCTGAGCGAGTTC
CAAGTGATTGTGCGCAAGACCAGCAGCAAGGCCGAAGAGATGGTGTTCGA
GATCGTGAAGTACCCCAGAATCTTCCGGGGCCACACACTGATCCAGAAAG
GCCTCGTGTGTGTGTCCGCCGAGAAGTTCGTGAAGTCTCCCGGCAAGGTG
CAGAGCGGCATGGACTACCTGTTCATCCCCACCTTTCTGAGCGTGACCTA
CTGTCCTGCCGCCATCAAGTTCCAGGTGCCAGGACCTATGCTGAAGATGC
GGAGCAGATACACCCAGTCTCTGCAGCTGGAACTGATGATCAGAATCCTG
TGCAAGCCCGACAGTCCCCTGATGAAGGTGCACATCCCCGACAAAGAAGG
CAGGGGCTGTCTCGTGTCTGTGTGGCTGCACGTGTGCAACATCTTCAAGA
GCGGCAACAAGAACGGCAGCGAGTGGCAAGAGTACTGGATGCGGAAGTGC
GCCAACATGCAGCTCGAAGTGTCTATCGCCGACATGTGGGGCCCTACCAT
CATCATCCACGCCAGAGGACACATCCCCAAGAGCGCCAAGCTGTTCTTTG
GCAAAGGCGGCTGGTCCTGCCATCCTCTGCATGAGGTTGTGCCCAGCGTG
ACCAAGACACTTTGGAGCGTGGGCTGCGAGATCACCAAGGCCAAGGCCAT
TATCCAAGAGAGCAGCATCTCCCTGCTGGTGGAAACCACAGACATCATTA
GCCCCAAAGTGAAGATCTCCAGCAAGCACAGAAGATTCGGCAAGAGCAAC
TGGGGCCTGTTTAAAAAGACCAAGAGCCTGCCTAACCTGACCGAGCTGGA
ATAG

Hence, preferably the fusion protein is encoded by a nucleic acid comprising a nucleotide sequence substantially as set out in SEQ ID NO: 8, or a variant or fragment thereof.

Advantageously, by combining the TMD and/or CT of MuV or PIV5 with viral antigens of a different virus, the inventors have enabled display of up to 2,000 viral fusion antigens on the surface of a VLP. This significantly increases the surface expression of viral antigens and therefore its valency, leading to a highly effective humoral immune response, the creation of significant numbers of antibodies, and therefore a robust vaccination to the viral antigen.

In a second aspect, there is provided a nucleic acid comprising a nucleotide sequence encoding the fusion protein according to the first aspect.

The nucleic acid may comprise DNA, RNA or a DNA/RNA hybrid sequence. Preferably, the nucleic acid comprises DNA or RNA. In one embodiment, the nucleic acid is a DNA sequence. In another embodiment, the nucleic acid is an RNA sequence. The RNA may be an mRNA sequence or a self-replicating RNA sequence (saRNA).

The skilled person would appreciate that self-amplifying mRNAs may contain the basic elements of mRNA (a cap, 5′ UTR, 3′UTR, and poly(A) tail of variable length), but may be considerably longer (for example 9-12 kb).

In one embodiment, the self-amplifying mRNAs is derived from an alphavirus genome, for example Sindbis, Semliki Forest, or Venezuelan equine encephalitis viruses. In this embodiment, the RNA self-amplifies using a replicase complex derived from the non-structural proteins including RNA dependent RNA polymerase, and advantageously yields higher protein expression of the encoded gene of interest than a similar dose of messenger RNA. In this embodiment, the nucleotide sequences encoding the fusion protein are inserted in place of the structural genes of the alphavirus downstream of a subgenomic promoter.

MuV and PIV5 have the advantage of displaying up to 2,000 viral fusion antigens on their surface and VLPs have previously been produced in mammalian cells by the co-expression of three proteins: glycoprotein, matrix protein and nucleoprotein (15-18). However, the inventors have now developed a novel platform for viral glycoprotein presentation on the surface of VLPs using modified MuV and PIV5 matrix proteins, that advantageously bypasses the need for the nucleoprotein to generate MuV and PIV5 pseudotyped VLPs, as in the prior art system.

The inventors have used Paramyxoviridea matrix proteins fused to a membrane targeting sequence as the core protein for the formation of VLPs. The matrix proteins of the Paramyxoviridea family do not contain membrane targeting sequences, and require interaction with viral and or host factors to assemble and bud from the membrane of eukaryotic cells. In the context of infectious virus, that lack of membrane targeting is a mechanism to prevent the release of non-infectious empty virions. Advantageously, the inventors have deliberately engineered membrane targeting to facilitate the efficient generation or VLPs solely using membrane targeted matrix protein to generate VLPs.

Thus, in a third aspect of the invention, there is provided a fusion protein suitable for forming a virus like particle (VLP), the fusion protein comprising a Paramyxovirus or Orthomyxovirus matrix protein and a membrane targeting signal (MTS).

The skilled person would understand that a virus like particle may refer to any vehicle that is capable of being decorated with an antigen, preferably a viral antigen. In particular, virus like particle may refer to a multiprotein structure that mimics the organization and conformation of native viruses, but which lacks the viral genome. Preferably, the VLP is an enveloped VLP.

Preferably, the Paramyxovirus or Orthomyxovirus is as defined in the first aspect.

Preferably, the fusion protein of the third aspect comprises a Paramyxovirus matrix protein. Preferably, the fusion protein comprises a MuV or PIV5 matrix protein.

The MTS may be disposed N-terminal or C-terminal of the Paramyxovirus or Orthomyxovirus matrix protein. Preferably, the Paramyxovirus or Orthomyxovirus matrix protein and MTS form a continuous amino acid sequence. Preferably, the MTS is disposed N-terminal of the Paramyxovirus matrix protein.

The MTS may be derived from a Fyn-like protein kinase, Lck-M, Src or C-YES. Hence, the MTS may be selected from the group consisting of: Fyn-like protein kinase-MGCVQCKDKE (SEQ ID No: 9); Lck-M-MGCGCSSHPE (SEQ ID No: 10), Src-MGSSKSKPKD (SEQ ID No: 11); and C-YES-MGCIKSKENK (SEQ ID No: 12), or a variant or fragment thereof.

Preferably, the MTS is derived from a Fyn-like protein kinase. Accordingly, in one embodiment, the MTS is provided herein as SEQ ID NO: 9, as follows:

[SEQ ID No: 9]
MGCVQCKDKE

Hence, preferably the MTS comprises an amino acid sequence substantially as set out in SEQ ID NO: 9, or a biologically active variant or fragment thereof.

As shown below, the fusion protein of the third aspect may comprise a MuV matrix protein (GenBank: D86171) and a MTS derived from a Fyn-like protein kinase (underlined). Accordingly, in one embodiment, the fusion protein is provided herein as SEQ ID NO: 13, as follows:

[SEQ ID No: 13]
MGCVQCKDKEAGSQIKIPLPKPPDSDSQRLNAFPVIMAQEGKGRLLRQIR
LRKILSGDPSDQQITFVNTYGFIRATPETSEFISESSQQKVTPVVTACML
SFGAGPVLEDPQHMLKALDQTDIRVRKTASDKEQILFEINRIPNLFRHHQ
ISADHLIQASSDKYVKSPAKLIAGVNYIYCVTFLSVTVCSASLKFRVARP
LLAARSRLVRAVQMEVLLRVTCKKDSQMAKSMLNDPDGEGCIASVWFHLC
NLCKGRNKLRSYDENYFASKCRKMNLTVSIGDMWGPTILVHAGGHIPTTA
KPFFNSRGWVCHPIHQSSPSLAKTLWSSGCEIKAASAILQGSDYASLAKT
DDIIYSKIKVDKDAANYKGVSWSPFRKSASMSNL*

Hence, preferably the fusion protein of the third aspect comprises an amino acid sequence substantially as set out in SEQ ID NO: 13, or a biologically active variant or fragment thereof.

In one embodiment, the fusion protein of the third aspect may be encoded by a nucleic acid having a nucleotide sequence comprising a Fyn proto-oncogene sequence (NCBI Reference Sequence NM_002037.5 and underlined), which is provided herein as SEQ ID NO: 14, as follows:

[SEQ ID No: 14]
ATGGGCTGTGTGCAATGTAAGGATAAAGAAGCTGGATCACAGATCAAAAT
TCCTCTTCCAAAGCCCCCCGATTCAGACTCTCAAAGATTAAATGCATTCC
CTGTAATCATGGCTCAAGAAGGCAAAGGACGACTCCTCAGACAAATCAGA
CTTAGGAAAATATTATCAGGGGATCCATCCGATCAGCAAATCACATTCGT
GAATACATATGGATTCATCCGTGCCACTCCAGAAACGTCCGAGTTCATCT
CTGAATCATCACAACAAAAGGTGACTCCTGTAGTGACGGCGTGTATGCTG
TCCTTCGGTGCTGGACCAGTCCTAGAAGACCCACAACATATGCTGAAAGC
TCTTGATCAGACAGATATCAGGGTTCGGAAGACAGCGAGTGACAAAGAGC
AGATCTTATTCGAGATCAACCGCATCCCCAATCTATTCAGGCATCATCAA
ATATCTGCGGACCATCTGATTCAGGCCAGTTCCGATAAATATGTCAAGTC
ACCAGCAAAGTTGATTGCAGGAGTAAATTACATCTACTGTGTCACATTTT
TATCCGTGACAGTTTGTTCCGCCTCACTCAAATTTCGGGTTGCGCGCCCA
TTGCTTGCTGCACGATCTAGATTAGTAAGAGCAGTTCAGATGGAAGTTTT
GCTTCGGGTAACTTGCAAAAAAGACTCCCAAATGGCAAAGAGCATGTTAA
ATGACCCTGATGGAGAAGGGTGCATTGCATCCGTGTGGTTCCACCTGTGT
AATCTGTGCAAAGGCAGGAATAAACTTAGAAGTTATGATGAAAATTATTT
TGCATCCAAGTGCCGTAAGATGAACCTGACAGTCAGCATAGGAGACATGT
GGGGACCAACCATTCTAGTCCATGCAGGCGGTCATATTCCGACAACTGCA
AAACCCTTTTTCAACTCAAGAGGCTGGGTTTGCCACCCCATCCACCAATC
ATCACCATCGTTGGCGAAGACCCTATGGTCATCTGGGTGTGAAATCAAGG
CTGCCAGTGCTATCCTCCAGGGCTCAGACTATGCATCACTTGCAAAAACT
GATGACATAATATATTCAAAGATAAAAGTTGATAAAGATGCAGCCAACTA
CAAAGGAGTATCCTGGAGTCCATTCAGGAAGTCTGCCTCAATGAGCAACC
TATGA

Hence, preferably the fusion protein of the third aspect is encoded by a nucleic acid sequence substantially as set out in SEQ ID NO: 14, or a variant or fragment thereof.

The nucleic acid sequence may be codon optimised for expression in humans. Accordingly, in one preferred embodiment, the fusion protein may be encoded by a nucleic acid having a nucleotide sequence, which is provided herein as SEQ ID NO: 15, as follows:

[SEQ ID No: 15]
ATGGGCTGCGTGCAGTGCAAGGACAAAGAGGCCGGCAGCCAGATCAAGA
TCCCCCTGCCCAAGCCCCCCGACAGCGATAGCCAGAGACTGAACGCCTT
CCCCGTGATCATGGCCCAGGAAGGCAAGGGCAGACTGCTGCGGCAGATC
CGGCTGAGAAAGATCCTGAGCGGCGACCCCAGCGACCAGCAGATCACCT
TCGTGAACACCTACGGCTTCATCCGGGCCACCCCCGAGACAAGCGAGTT
CATCAGCGAGAGCAGCCAGCAGAAAGTGACCCCCGTCGTGACCGCCTGC
ATGCTGTCTTTTGGAGCCGGCCCTGTGCTGGAAGATCCCCAGCACATGC
TGAAGGCCCTGGACCAGACCGACATCAGAGTGCGCAAGACCGCCAGCGA
CAAAGAGCAGATCCTGTTCGAGATCAACCGCATCCCCAACCTGTTCCGG
CACCACCAGATCAGCGCCGACCACCTGATTCAGGCCAGCTCCGACAAAT
ACGTGAAGTCCCCCGCCAAGCTGATCGCCGGCGTGAACTATATCTACTG
CGTGACCTTCCTGAGCGTGACCGTGTGCAGCGCCAGCCTGAAGTTCAGA
GTGGCCAGACCTCTGCTGGCCGCCAGATCTAGACTCGTGCGGGCCGTGC
AGATGGAAGTGCTGCTGAGAGTGACCTGCAAGAAAGACAGCCAGATGGC
CAAGAGCATGCTGAACGACCCCGACGGCGAGGGCTGTATCGCCAGCGTG
TGGTTCCACCTGTGCAATCTGTGCAAGGGCCGGAACAAGCTGCGGAGCT
ACGACGAGAACTACTTCGCCAGCAAGTGCCGGAAGATGAACCTGACCGT
GTCCATCGGCGACATGTGGGGCCCTACCATCCTGGTGCATGCCGGCGGA
CACATCCCTACCACCGCCAAGCCATTCTTCAACAGCCGGGGCTGGGTGT
GCCACCCCATCCATCAGTCTAGCCCCAGCCTGGCCAAGACCCTGTGGTC
TAGCGGCTGCGAGATCAAGGCCGCCTCTGCCATCCTGCAGGGCAGCGAT
TATGCCTCCCTGGCCAAAACCGACGACATCATCTACAGCAAGATCAAGG
TGGACAAGGACGCCGCCAACTACAAGGGAGTGTCTTGGAGCCCCTTCAG
AAAGTCCGCCAGCATGAGCAACCTGTAA

Hence, preferably the fusion protein is encoded by nucleic acid comprising a nucleotide sequence substantially as set out in SEQ ID NO: 15, or a variant or fragment thereof.

In another embodiment, the fusion protein of the third aspect may be encoded by a nucleic acid having a nucleotide sequence, which is provided herein as SEQ ID NO: 16, as follows:

[SEQ ID No: 16]
ATGGGCTGTGTGCAGTGCAAGGACAAAGAGGCCGGCAGCCAGATCAAGA
TCCCTCTGCCTAAGCCTCCTGACAGCGACAGCCAGAGACTGAACGCCTT
TCCTGTGATCATGGCCCAAGAAGGCAAGGGCAGACTGCTGCGGCAGATC
CGGCTGAGAAAGATCCTGAGCGGCGACCCTAGCGACCAGCAGATCACCT
TCGTGAACACCTACGGCTTCATCCGGGCCACACCTGAGACAAGCGAGTT
CATCAGCGAGAGCAGCCAGCAGAAAGTGACCCCTGTGGTCACCGCCTGC
ATGCTGTCTTTTGGAGCCGGACCTGTGCTGGAAGATCCCCAGCACATGC
TGAAGGCCCTGGACCAGACCGACATCAGAGTGCGGAAAACCGCCAGCGA
CAAAGAGCAGATCCTGTTCGAGATCAACAGAATCCCCAACCTGTTCCGG
CACCACCAGATCTCTGCCGACCATCTGATTCAGGCCAGCTCCGACAAAT
ACGTGAAGTCCCCTGCCAAGCTGATCGCCGGCGTGAACTATATCTACTG
CGTGACCTTCCTGAGCGTGACCGTGTGTAGCGCCAGCCTGAAGTTCAGA
GTGGCCAGACCTCTGCTGGCCGCCAGAAGCAGACTTGTTAGAGCCGTGC
AGATGGAAGTGCTGCTGAGAGTGACCTGCAAGAAAGACTCCCAGATGGC
CAAGAGCATGCTGAACGACCCTGATGGCGAGGGCTGTATCGCCAGCGTG
TGGTTCCACCTGTGCAATCTGTGCAAAGGCCGGAACAAGCTGCGGAGCT
ACGACGAGAATTACTTCGCCAGCAAGTGCCGGAAGATGAACCTGACCGT
GTCCATCGGCGATATGTGGGGCCCTACAATCCTGGTGCATGCCGGCGGA
CACATCCCTACAACCGCCAAGCCATTCTTCAACTCCAGAGGCTGGGTCT
GCCATCCTATCCACCAGTCTAGCCCCAGCCTGGCCAAGACACTTTGGAG
CAGCGGATGCGAGATCAAGGCCGCCTCTGCTATCCTGCAGGGCAGCGAT
TATGCCTCTCTGGCCAAAACCGACGACATCATCTACAGCAAGATCAAGG
TGGACAAGGACGCCGCCAACTACAAGGGAGTCAGCTGGTCCCCATTCCG
GAAGTCTGCCAGCATGAGCAACCTGTAA

Hence, preferably the fusion protein of the third aspect is encoded by a nucleic acid comprising a nucleotide sequence substantially as set out in SEQ ID NO: 16, or a variant or fragment thereof.

The fusion protein of the third aspect may comprise a PIV5 matrix protein and a MTS derived from a Fyn-like protein kinase (underlined). Thus, in one embodiment, the fusion protein is provided herein as SEQ ID NO: 17, as follows:

[SEQ ID No: 17]
MGCVQCKDKEPSISIPADPTNPRQSIKAFPIVINSDGGEKGRLVKQLRT
TYLNDLDTHEPLVTFVNTYGFIYEQDRGNTIVGEDQLGKKREAVTAAMV
TLGCGPNLPSLGNVLGQLSEFQVIVRKTSSKAEEMVFEIVKYPRIFRGH
TLIQKGLVCVSAEKFVKSPGKVQSGMDYLFIPTFLSVTYCPAAIKFQVP
GPMLKMRSRYTQSLQLELMIRILCKPDSPLMKVHIPDKEGRGCLVSVWL
HVCNIFKSGNKNGSEWQEYWMRKCANMQLEVSIADMWGPTIIIHARGHI
PKSAKLFFGKGGWSCHPLHEVVPSVTKTLWSVGCEITKAKAIIQESSIS
LLVETTDIISPKVKISSKHRRFGKSNWGLFKKTKSLPNLTELE*

Hence, preferably the fusion protein of the third aspect comprises an amino acid sequence substantially as set out in SEQ ID NO: 17, or a biologically active variant or fragment thereof.

In one embodiment, the fusion protein of the third aspect may be encoded by a nucleic acid having a nucleotide sequence, which is provided herein as SEQ ID NO: 18, as follows:

[SEQ ID No: 18]
ATGGGCTGTGTGCAATGTAAGGATAAAGAACCATCCATCAGCATCCCCG
CAGACCCCACCAATCCACGTCAATCAATAAAAGCGTTCCCAATTGTGAT
CAACAGTGATGGGGGTGAGAAAGGCCGCTTGGTTAAACAACTACGCACA
ACCTACTTGAATGACCTAGATACTCATGAGCCACTGGTGACATTCGTAA
ATACCTATGGATTCATCTACGAACAGGATCGGGGGAATACTATTGTCGG
AGAGGATCAACTTGGGAAGAAAAGAGAGGCTGTGACTGCTGCAATGGTT
ACCCTTGGATGTGGGCCTAATCTACCATCATTAGGGAATGTCCTGGGAC
AACTGAGTGAATTCCAGGTCATTGTTAGGAAGACATCCAGCAAAGCGGA
AGAGATGGTCTTTGAAATTGTTAAGTATCCGAGAATATTTCGGGGTCAT
ACATTAATCCAGAAAGGACTAGTCTGTGTCTCCGCAGAAAAATTTGTTA
AGTCACCAGGGAAAGTACAATCTGGAATGGACTATCTCTTCATTCCGAC
ATTTCTGTCAGTGACTTACTGTCCAGCTGCAATCAAATTTCAGGTACCT
GGCCCCATGTTGAAAATGAGATCAAGATACACTCAGAGCTTACAACTTG
AACTAATGATAAGAATCCTGTGTAAGCCCGATTCGCCACTTATGAAGGT
CCATATCCCTGACAAGGAAGGAAGAGGATGTCTTGTATCAGTATGGCTG
CATGTATGCAACATCTTCAAATCAGGAAACAAGAATGGCAGTGAGTGGC
AGGAATACTGGATGAGAAAGTGTGCCAACATGCAACTTGAAGTGTCGAT
TGCAGATATGTGGGGACCAACTATCATAATTCATGCCAGAGGTCACATT
CCCAAAAGTGCTAAGTTGTTTTTTGGAAAGGGTGGATGGAGCTGCCATC
CACTTCACGAAGTTGTTCCAAGTGTCACTAAAACACTATGGTCCGTGGG
CTGTGAGATTACAAAGGCGAAGGCAATAATACAAGAGAGTAGCATCTCT
CTTATCGTGGAGACTACTGACATCATAAGTCCAAAAGTCAAAATTTCAT
CTAAGCATCGCCGCTTTGGGAAATCAAATTGGGGTCTGTTCAAGAAAAC
TAAATCACTGCCTAACCTGACGGAGCTGGAATGA

Hence, preferably the fusion protein of the third aspect may be encoded by a nucleic acid comprising a nucleotide sequence substantially as set out in SEQ ID NO: 18, or a variant or fragment thereof.

The nucleotide sequence may be codon optimised for expression in humans. Accordingly, in one embodiment, the fusion protein may be encoded by a nucleic acid having a nucleotide sequence, which is provided herein as SEQ ID NO: 19, as follows:

[SEQ ID No: 19]
ATGGGCTGCGTGCAGTGCAAGGACAAAGAGCCCAGCATCAGCATCCCCG
CCGACCCCACCAATCCCCGGCAGAGCATTAAGGCCTTCCCCATCGTGAT
CAACAGCGACGGCGGCGAGAAGGGCCGGCTCGTGAAACAGCTGAGAACC
ACCTACCTGAACGACCTGGACACCCACGAGCCCCTCGTGACCTTCGTGA
ACACCTACGGCTTCATCTACGAGCAGGACCGGGGCAACACCATCGTGGG
CGAAGATCAGCTGGGCAAGAAACGCGAGGCCGTGACAGCCGCCATGGTC
ACACTGGGCTGTGGCCCCAATCTGCCCTCCCTGGGAAATGTGCTGGGCC
AGCTGAGCGAGTTCCAAGTGATCGTGCGCAAGACCAGCAGCAAGGCCGA
GGAAATGGTGTTCGAGATCGTGAAGTACCCCCGGATCTTCCGGGGCCAC
ACCCTGATCCAGAAAGGCCTCGTGTGTGTGTCCGCCGAGAAGTTTGTGA
AGTCCCCTGGCAAGGTGCAGAGCGGCATGGACTACCTGTTCATCCCCAC
CTTTCTGAGCGTGACCTACTGCCCTGCCGCCATCAAGTTCCAGGTGCCA
GGCCCCATGCTGAAGATGCGGAGCAGATACACCCAGAGCCTGCAGCTGG
AACTGATGATCAGAATCCTGTGCAAGCCCGACAGCCCCCTGATGAAGGT
GCACATCCCCGACAAAGAGGGCAGAGGCTGCCTGGTGTCTGTGTGGCTG
CACGTGTGCAACATCTTCAAGAGCGGCAACAAGAACGGCAGCGAGTGGC
AGGAATACTGGATGCGGAAGTGCGCCAACATGCAGCTGGAAGTGTCTAT
CGCCGACATGTGGGGCCCTACCATCATCATCCACGCCAGAGGCCACATC
CCCAAGAGCGCCAAGCTGTTCTTTGGCAAGGGCGGCTGGTCCTGCCACC
CTCTGCATGAGGTGGTGCCCTCCGTGACCAAGACCCTGTGGAGCGTGGG
CTGCGAGATCACCAAGGCCAAGGCCATCATCCAGGAAAGCAGCATCTCC
CTGCTGGTGGAAACCACCGACATCATCAGCCCCAAAGTGAAGATCTCCA
GCAAGCACAGAAGATTCGGCAAGAGCAACTGGGGCCTGTTCAAAAAGAC
CAAGAGCCTGCCCAACCTGACCGAGCTGGAGTAA

Hence, preferably the fusion protein of the third aspect may be encoded by a nucleic acid comprising a nucleotide sequence substantially as set out in SEQ ID NO: 19, or a variant or fragment thereof.

In one embodiment, the fusion protein of the third aspect may be encoded by a nucleic acid having a nucleotide sequence, which is provided herein as SEQ ID NO: 20, as follows:

[SEQ ID No: 20]
ATGGGCTGTGTGCAGTGCAAGGACAAAGAGCCCAGCATCAGCATCCCCG
CCGATCCTACCAATCCTCGGCAGAGCATCAAGGCCTTTCCAATCGTGAT
CAACAGCGACGGCGGCGAGAAGGGCAGACTGGTTAAGCAGCTGAGAACC
ACCTACCTGAACGACCTGGACACCCACGAGCCTCTGGTCACCTTCGTGA
ACACCTACGGCTTCATCTACGAGCAGGACCGGGGCAATACCATCGTGGG
CGAAGATCAGCTGGGCAAGAAACGGGAAGCCGTGACAGCCGCCATGGTC
ACACTTGGCTGTGGCCCTAATCTGCCTAGCCTGGGCAATGTGCTGGGCC
AGCTGAGCGAGTTCCAAGTGATCGTGCGGAAAACCAGCAGCAAGGCCGA
GGAAATGGTGTTCGAGATCGTGAAGTACCCCAGAATCTTCCGGGGCCAC
ACACTGATCCAGAAAGGCCTCGTGTGTGTGTCCGCCGAGAAGTTCGTGA
AGTCTCCCGGCAAGGTGCAGAGCGGCATGGACTACCTGTTCATCCCCAC
CTTTCTGAGCGTGACCTACTGTCCTGCCGCCATCAAGTTCCAGGTGCCA
GGACCTATGCTGAAGATGCGGAGCAGATACACACAGAGCCTGCAGCTGG
AACTGATGATCAGAATCCTGTGCAAGCCAGACAGCCCTCTGATGAAGGT
GCACATCCCCGACAAAGAAGGCAGAGGCTGCCTGGTGTCTGTGTGGCTG
CACGTGTGCAACATCTTCAAGAGCGGCAACAAGAACGGCAGCGAGTGGC
AAGAGTACTGGATGCGGAAGTGCGCCAACATGCAGCTCGAAGTGTCTAT
CGCCGACATGTGGGGCCCTACCATCATCATCCACGCCAGAGGACACATC
CCCAAGAGCGCCAAGCTGTTCTTTGGCAAAGGCGGCTGGTCCTGCCATC
CTCTGCATGAGGTTGTGCCCAGCGTGACCAAGACACTTTGGAGCGTGGG
CTGCGAGATCACCAAGGCCAAGGCCATCATCCAAGAGAGCAGCATCTCC
CTGCTGGTGGAAACCACCGACATCATCAGCCCCAAAGTGAAGATCTCCA
GCAAGCACAGAAGATTCGGCAAGAGCAACTGGGGCCTGTTCAAAAAGAC
CAAGAGCCTGCCTAACCTGACCGAGCTGGAATAA

Hence, preferably the fusion protein of the third aspect may be encoded by a nucleic acid comprising a nucleotide sequence substantially as set out in SEQ ID NO: 20, or a variant or fragment thereof.

In a fourth aspect, there is provided a nucleic acid sequence comprising a nucleotide sequence encoding the fusion protein according to the third aspect.

The nucleic acid sequence may be a DNA, RNA or DNA/RNA hybrid sequence. Preferably the nucleotide sequence is a DNA or RNA sequence. In one embodiment, the nucleic acid sequence is a DNA sequence. In another embodiment the nucleic acid sequence is an RNA sequence. In one embodiment, the RNA may be an mRNA sequence or a self-replicating RNA sequence.

In a fifth aspect, there is provided a virus like particle (VLP) comprising the fusion protein according to the third aspect.

The average diameter of the VLP of the third aspect may be between 30 nm and 1000 nm, 40 um and 900 nm, 50 nm and 800 nm, 60 nm and 700 nm, 70 nm and 600 nm, 80 nm and 500 nm, 90 nm and 400 nm, 100 nm and 300 nm. Preferably, the average diameter is between 30 nm and 1000 nm, 900 nm, 800 nm, 700 nm, 600 nm, 500 nm, 400 nm, 300 nm, 200 nm or 100 nm. Preferably, the average diameter is between 40 nm and 1000 nm, 900 nm, 800 nm, 700 nm, 600 nm, 500 nm, 400 nm, 300 nm, 200 nm or 100 nm. Preferably, the average diameter is between 50 nm and 1000 nm, 900 nm, 800 nm, 700 nm, 600 nm, 500 nm, 400 nm, 300 nm, 200 nm or 100 nm. Preferably, the average diameter is between 60 nm and 1000 nm, 900 nm, 800 nm, 700 nm, 600 nm, 500 nm, 400 nm, 300 nm, 200 nm or 100 nm. Preferably, the average diameter is between 70 nm and 1000 nm, 900 nm, 800 nm, 700 nm, 600 nm, 500 nm, 400 nm, 300 nm, 200 nm or 100 nm. Preferably, the average diameter is between 80 nm and 100 nm, 900 nm, 800 nm, 700 nm, 600 nm, 500 nm, 400 nm, 300 nm, 200 nm or 100 nm. Preferably, the average diameter is between 90 nm and 1000 nm, 900 nm, 800 nm, 700 nm, 600 nm, 500 nm, 400 nm, 300 nm, 200 nm or 100 nm. Preferably, the average diameter is between 100 nm and 1000 nm, 900 nm, 800 nm, 700 nm, 600 nm, 500 nm, 400 nm, 300 nm, 200 nm.

The skilled person would appreciate that the diameter of a VLP may be determined using: Nanoparticle Tracking Analysis; Tunable Resistive Pulse Sensing (TRPS) or dynamic light scattering, techniques that allow high-throughput single particle measurements as colloids and/or biomolecular analytes. Preferably, the VLP is an enveloped VLP.

In a sixth aspect, there is provided a method of producing a virus like particle (VLP) according to the fifth aspect, the method comprising expressing a nucleic acid of the fourth aspect in a host cell.

The host cell may be a eukaryotic or prokaryotic host cell. Preferably, the host cell is a eukaryotic host cell. More preferably, the host cell is a mammalian host cell such as Human embryonic kidney 293 cells or Chinese hamster ovary (CHO) cells. Cells may be co-transduced with DNA sequences encoding a fusion protein suitable for forming a virus like particle (VLP) and matrix-MTS protein to generate VLPs secreted in the supernatant. This may be performed by transient transfection or through the establishment of stable cells lines expressing both fusion protein and matrix MTS. The supernatants containing the VLPs may be harvested, and VLPs purified from cell according to standard processes for virus or VLP purification, which would be known to those skilled in the art.

In a seventh aspect of the invention, there is provided a fusion protein suitable for forming a virus like particle (VLP) displaying an antigen, wherein the fusion protein comprises the fusion protein of the first aspect and the fusion protein of the third aspect.

Any of the fusion proteins described herein may be isolated. The fusion proteins described herein may be purified, preferably to at least 90%, 91%, 92%, 93%, 94%, 95%, 96%, 97%, 98%, 99% or 100% purity.

The fusion protein of the first aspect may be disposed C-terminal of the fusion protein of the third aspect. However, preferably the fusion protein of the first aspect is disposed N-terminal of the fusion protein of the third aspect. In one embodiment, the fusion protein of the first aspect and the fusion protein of the third aspect may be linked to one another with a cleavable spacer sequence. The spacer sequence is configured to be digested or cleaved to thereby produce the two fusion proteins as separate molecules. As such, the spacer sequence is preferably a cleavable peptide, preferably a 2A peptide.

Suitable 2A peptides include the porcine teschovirus-12A (P2A)-ATNFSLLKQAGDVEENPGP (SEQ ID No: 28), thosea asigna virus 2A (T2A)-QCTNYALLKLAGDVESNPGP (SEQ ID No: 29), equine rhinitis A virus 2A (E2A), and Foot and mouth disease virus 2A (F2A) VKQTLNFDLLKLAGDVESNPGP (SEQ ID No: 30). Preferably, the 2A peptide is thosea asigna virus 2A (T2A).

In another embodiment, the cleavable peptide is a self-cleaving peptide. Preferably, the self-cleaving peptide is a furin/2A peptide. The furin sequence may be disposed 3′ or 5′ of the 2A sequence. Preferably, the furin sequence is disposed 5′ of the 2A sequence, and preferably with a GSG spacer disposed between the furin and 2A sequence.

The skilled person would appreciate that furin is a ubiquitous calcium-dependent proprotein convertase located in the secretory pathway (mainly in the golgi and trans-golgi network) that cleaves precursor proteins at a specific recognition sequence-canonically R-X-R/K/X-R (SEQ ID No: 26), and cleaving the proprotein after the final R. Thus, in one embodiment the furin sequence is R-X-R/K/X-R. However, preferably, the furin sequence is the optimised sequence RRRRRR (SEQ ID No: 27) a GSG sequence. In another embodiment, the furin sequence is R-X-X-R (SEQ ID No: 35), wherein X is any amino acid. In another embodiment, the furin sequence is R-X₁-X₂-R (SEQ ID No: 36), wherein X₁ is any amino acid and X₂ is R or K. In a preferred embodiment, the furin sequence is R-R-R-R (SEQ ID No: 37). Preferably, the GSG spacer is disposed 3′ of the furin sequence and 5′ of the 2A sequence.

Thus, preferably, the spacer sequence is the furin/T2A, as provided by NCBI Reference Sequence: GenBank: AAC97195.1, and provided herein as SEQ ID No: 21, as follows:

[SEQ ID No: 21]
RRRRRRGSGEGRGSLLTCGDVEENPGP

Hence, preferably the spacer sequence comprises an amino acid sequence substantially as set out in SEQ ID NO: 17, or a variant or fragment thereof.

Accordingly, in one embodiment, the fusion protein of the fifth aspect may comprise a MuV matrix protein, a MTS derived from a Fyn-like protein kinase (bold), a MuV TMD and CT (bold underlined) fused to HIV antigen (e.g. HIV-1 Env) and a furin/T2A sequence (underlined), and is provided herein as SEQ ID NO: 22, as follows:

[SEQ ID No: 22]
MDRAKLLLLLLLLLLPQAQAVENLWVTVYYGVPVWKDAETTLFCASDAK
AYDTEVRNVWATHACVPTDPNPQEIVLENVTENFNMWKNNMVEQMHTDI
ISLWDQSLKPCVKLTPLCVTLNCTNVNVTNTTNNTEEKGEIKNCSFNIT
TELRDKKKKVYALFYRLDVVPIDDNNNNSSNYRLINCNTSAITQACPKV
SFEPIPIHYCAPAGFAILKCNDKKFNGTGPCKNVSTVQCTHGIKPVVST
QLLLNGSLAEEEIIIRSENITNNAKTIIVQLNESVEINCTRPNNNTRKS
IRIGPGQWFYATGDIIGDIRQAHCNISGTKWNKTLQQVVKKLREHFNNK
TIIFNPSSGGDLEITTHSFNCGGEFFYCNTSGLFNSTWIGNGTKNNNNT
NDTITLPCRIKQIINMWQRVGQPMYAPPIQGKIRCVSNITGLLLTRDGG
NNNTNETETFRPGGGDMRDNWRSELYKYKVVKIEPLGVAPTRCKRRVVE
GGGGSGGGGSAVGIGAVFLGFLGAAGSTMGAASMTLTVQARNLLSGGSG
SGSGSTVWGIKQLQARVLAVERYLRDQQLLGIWGCSGKLICCTNVPWNS
SWSNKSQDEIWDNMTWMEWDKEINNYTDIIYSLIEESQNQQEKNEQDLL
ALDKWASLWNWFDITNWLWYIKAIIVAALVLSILSIIISLLFCCWAYVA
TKEIRRINFKTNHINTISSSVDDLIRYRRRRRRGSGEGRGSLLTCGDVE
ENPGPMGCVQCKDKEAGSQIKIPLPKPPDSDSQRLNAFPVIMAQEGKGR
LLRQIRLRKILSGDPSDQQITFVNTYGFIRATPETSEFISESSQQKVTP
VVTACMLSFGAGPVLEDPQHMLKALDQTDIRVRKTASDKEQILFEINRI
PNLFRHHQISADHLIQASSDKYVKSPAKLIAGVNYIYCVTFLSVTVCSA
SLKFRVARPLLAARSRLVRAVQMEVLLRVTCKKDSQMAKSMLNDPDGEG
CIASVWFHLCNLCKGRNKLRSYDENYFASKCRKMNLTVSIGDMWGPTIL
VHAGGHIPTTAKPFFNSRGWVCHPIHQSSPSLAKTLWSSGCEIKAASAI
LQGSDYASLAKTDDIIYSKIKVDKDAANYKGVSWSPFRKSASMSNL*

Hence, preferably the fusion protein of the seventh aspect comprises an amino acid sequence substantially as set out in SEQ ID NO: 22, or a variant or fragment thereof.

In one embodiment, the fusion protein of the seventh aspect may be encoded by a nucleic acid having a nucleotide sequence, which is provided herein as SEQ ID NO: 23, as follows:

[SEQ ID No: 23]
ATGGACAGAGCCAAACTGCTGCTGCTCCTGTTGCTCCTCCTGCTGCCTC
AGGCTCAGGCCGTGGAAAATCTGTGGGTCACCGTGTACTACGGCGTGCC
CGTGTGGAAGGATGCCGAGACAACACTGTTCTGTGCCAGCGACGCCAAG
GCCTACGATACCGAAGTGCGGAATGTGTGGGCCACTCACGCCTGCGTTC
CCACCGATCCTAATCCTCAAGAGATCGTGCTGGAAAACGTGACCGAGAA
CTTCAACATGTGGAAGAACAACATGGTCGAGCAGATGCACACCGACATC
ATCAGCCTGTGGGACCAGAGCCTGAAGCCTTGCGTGAAGCTGACCCCTC
TGTGCGTGACCCTGAACTGCACCAACGTGAACGTGACCAACACCACCAA
CAACACCGAGGAAAAGGGCGAGATCAAGAACTGCAGCTTCAACATCACC
ACCGAGCTGCGGGACAAGAAAAAGAAGGTGTACGCCCTGTTCTACCGGC
TGGACGTGGTGCCCATCGACGATAACAACAACAACTCCAGCAATTACCG
GCTGATCAACTGCAACACCAGCGCCATCACTCAGGCCTGTCCTAAGGTG
TCCTTCGAGCCCATTCCTATCCACTACTGTGCCCCTGCCGGCTTCGCCA
TCCTGAAGTGCAACGACAAGAAGTTCAACGGCACAGGCCCCTGCAAGAA
CGTGTCCACCGTGCAGTGTACCCACGGCATCAAGCCAGTGGTGTCTACC
CAGCTGCTGCTGAATGGCTCTCTGGCCGAGGAAGAGATCATCATCAGAA
GCGAGAACATCACGAACAACGCCAAGACCATCATCGTGCAGCTGAACGA
GAGCGTGGAAATCAATTGCACCCGGCCTAACAACAATACCCGGAAGTCC
ATCAGAATCGGCCCTGGCCAGTGGTTTTATGCCACCGGCGATATTATCG
GCGACATCAGACAGGCCCACTGTAACATCAGCGGCACCAAGTGGAACAA
GACCCTGCAGCAGGTCGTGAAGAAGCTGAGAGAGCACTTCAACAACAAG
ACGATCATCTTCAACCCCAGCTCTGGCGGCGACCTGGAAATCACCACAC
ACAGCTTCAATTGTGGCGGCGAGTTCTTCTACTGCAATACCTCCGGCCT
GTTCAACAGCACCTGGATCGGCAATGGCACCAAGAACAACAACAACACC
AACGACACCATCACACTGCCCTGCCGGATCAAGCAGATCATCAATATGT
GGCAGCGCGTGGGCCAGCCTATGTACGCTCCTCCAATCCAGGGCAAGAT
CAGATGCGTGTCCAATATCACCGGCCTGCTGCTCACAAGAGATGGCGGA
AACAACAACACGAATGAGACAGAGACATTCAGACCCGGCGGAGGCGACA
TGAGAGACAATTGGAGAAGCGAGCTGTACAAGTACAAGGTGGTCAAGAT
CGAGCCCCTGGGCGTCGCACCTACACGGTGCAAAAGAAGAGTGGTCGAA
GGCGGCGGAGGAAGCGGAGGCGGAGGATCTGCTGTTGGAATCGGAGCCG
TGTTCCTGGGCTTTCTGGGAGCCGCTGGATCTACAATGGGCGCTGCCAG
CATGACCCTGACAGTGCAGGCTAGAAATCTGCTGTCTGGCGGCAGCGGC
TCTGGCTCAGGATCTACAGTGTGGGGAATCAAGCAGCTGCAGGCCAGAG
TGCTGGCCGTCGAAAGATACCTGAGAGATCAGCAGCTCCTCGGCATCTG
GGGCTGTTCTGGCAAGCTGATCTGCTGCACCAATGTGCCCTGGAACAGC
TCCTGGTCCAACAAGAGCCAGGACGAGATCTGGGACAACATGACCTGGA
TGGAATGGGACAAAGAGATTAACAACTATACGGACATCATCTACAGCCT
GATCGAGGAAAGCCAGAACCAGCAAGAGAAGAACGAGCAGGACCTGCTG
GCCCTGGATAAGTGGGCTAGCCTGTGGAATTGGTTCGACATCACCAACT
GGCTGTGGTACATCAAGGCCATCATTGTGGCCGCTCTGGTGCTGAGCAT
CCTGTCCATCATCATCTCCCTGCTGTTCTGCTGCTGGGCCTACGTGGCC
ACCAAAGAGATCAGACGGATCAACTTCAAGACCAACCACATCAACACCA
TCAGCTCCAGCGTGGACGACCTGATCAGATACCGGCGGAGAAGAAGAAG
AGGCTCCGGCGAAGGCAGAGGCAGCCTTCTTACATGTGGCGACGTGGAA
GAGAACCCCGGACCTATGGGATGCGTGCAGTGCAAAGACAAAGAGGCCG
GCAGCCAGATCAAGATCCCTCTGCCTAAGCCTCCTGACAGCGACAGCCA
GAGACTGAACGCTTTCCCCGTGATCATGGCCCAAGAAGGCAAGGGCAGA
CTGCTGCGGCAGATCCGGCTGAGAAAGATCCTCAGCGGCGACCCTAGCG
ACCAGCAGATTACCTTCGTGAACACCTACGGCTTCATCCGGGCCACACC
TGAGACAAGCGAGTTCATCAGCGAGAGCAGCCAGCAGAAAGTGACCCCT
GTGGTCACCGCCTGCATGCTGTCTTTTGGAGCCGGACCTGTGCTGGAAG
ATCCCCAGCACATGCTGAAAGCCCTGGACCAGACAGACATCAGAGTGCG
CAAGACCGCCAGCGACAAAGAGCAGATTCTGTTCGAGATCAACAGGATT
CCCAACCTGTTCCGGCACCACCAGATCAGCGCCGATCATCTGATTCAGG
CCAGCTCCGACAAATACGTGAAGTCCCCTGCCAAGCTGATTGCCGGCGT
GAACTATATCTACTGCGTGACCTTCCTGAGCGTGACCGTGTGTAGCGCC
TCTCTGAAGTTTAGAGTGGCCAGACCTCTGCTGGCCGCCAGATCCAGAC
TTGTTAGAGCCGTGCAGATGGAAGTGCTGCTGAGAGTGACCTGCAAAAA
GGACTCCCAGATGGCCAAGAGCATGCTGAACGACCCTGATGGCGAGGGC
TGTATCGCCAGCGTGTGGTTCCACCTGTGCAATCTGTGCAAAGGCCGGA
ACAAGCTGCGGAGCTACGACGAGAATTACTTCGCCAGCAAGTGCCGGAA
GATGAACCTGACCGTGTCCATCGGCGATATGTGGGGCCCTACAATCCTG
GTGCATGCCGGCGGACACATCCCTACAACCGCCAAGCCATTCTTCAACT
CCAGAGGCTGGGTCTGCCATCCAATCCACCAGTCTAGTCCCAGCCTGGC
CAAGACACTGTGGTCTAGCGGCTGCGAAATCAAAGCCGCCAGCGCTATC
CTGCAGGGCTCTGATTATGCCTCTCTGGCTAAGACCGACGACATTATCT
ACTCCAAGATCAAGGTGGACAAGGACGCCGCCAACTACAAGGGAGTCAG
CTGGTCCCCATTCAGAAAGTCCGCCAGCATGTCCAACCTGTAG

Hence, preferably the fusion protein of the seventh aspect may be encoded by a nucleic acid comprising a nucleotide sequence substantially as set out in SEQ ID NO: 23, or a variant or fragment thereof.

In another embodiment, the fusion protein of the seventh aspect may comprise a PIV5 matrix protein, a MTS derived from a Fyn-like protein kinase (bold), a PIV5 TMD and CT (bold underlined) fused to HIV antigen (e.g. HIV-1 Env) and a T2A sequence (underlined), having an amino acid sequence which is provided herein as SEQ ID NO: 24, as follows:

[SEQ ID No: 24]
MDRAKLLLLLLLLLLPQAQAVENLWVTVYYGVPVWKDAETTLFCASDAK
AYDTEVRNVWATHACVPTDPNPQEIVLENVTENFNMWKNNMVEQMHTDI
ISLWDQSLKPCVKLTPLCVTLNCTNVNVTNTTNNTEEKGEIKNCSFNIT
TELRDKKKKVYALFYRLDVVPIDDNNNNSSNYRLINCNTSAITQACPKV
SFEPIPIHYCAPAGFAILKCNDKKFNGTGPCKNVSTVQCTHGIKPVVST
QLLLNGSLAEEEIIIRSENITNNAKTIIVQLNESVEINCTRPNNNTRKS
IRIGPGQWFYATGDIIGDIRQAHCNISGTKWNKTLQQVVKKLREHFNNK
TIIFNPSSGGDLEITTHSFNCGGEFFYCNTSGLFNSTWIGNGTKNNNNT
NDTITLPCRIKQIINMWQRVGQPMYAPPIQGKIRCVSNITGLLLTRDGG
NNNTNETETFRPGGGDMRDNWRSELYKYKVVKIEPLGVAPTRCKRRVVE
GGGGSGGGGSAVGIGAVFLGFLGAAGSTMGAASMTLTVQARNLLSGGSG
SGSGSTVWGIKQLQARVLAVERYLRDQQLLGIWGCSGKLICCTNVPWNS
SWSNKSQDEIWDNMTWMEWDKEINNYTDIIYSLIEESQNQQEKNEQDLL
ALDKWASLWNWFDITNWLWYIKVLSIIAICLGSLGLILIILLSVVVWKL
LTIVVANRNRMENFVYHKRRRRRRGSGEGRGSLLTCGDVEENPGPMGCV
QCKDKEPSISIPADPTNPRQSIKAFPIVINSDGGEKGRLVKQLRTTYLN
DLDTHEPLVTFVNTYGFIYEQDRGNTIVGEDQLGKKREAVTAAMVTLGC
GPNLPSLGNVLGQLSEFQVIVRKTSSKAEEMVFEIVKYPRIFRGHTLIQ
KGLVCVSAEKFVKSPGKVQSGMDYLFIPTFLSVTYCPAAIKFQVPGPML
KMRSRYTQSLQLELMIRILCKPDSPLMKVHIPDKEGRGCLVSVWLHVCN
IFKSGNKNGSEWQEYWMRKCANMQLEVSIADMWGPTIIIHARGHIPKSA
KLFFGKGGWSCHPLHEVVPSVTKTLWSVGCEITKAKAIIQESSISLLVE
TTDIISPKVKISSKHRRFGKSNWGLFKKTKSLPNLTELE

Hence, preferably the fusion protein of the seventh aspect comprises an amino acid sequence substantially as set out in SEQ ID NO: 18, or a variant or fragment thereof.

In one embodiment, the fusion protein of the seventh aspect may be encoded by a nucleic acid having a nucleotide sequence, which is provided herein as SEQ ID NO: 25, as follows:

[SEQ ID No: 25]
ATGGACAGAGCCAAACTGCTGCTGCTCCTGTTGCTCCTCCTGCTGCCTC
AGGCTCAGGCCGTGGAAAATCTGTGGGTCACCGTGTACTACGGCGTGCC
CGTGTGGAAGGATGCCGAGACAACACTGTTCTGTGCCAGCGACGCCAAG
GCCTACGATACCGAAGTGCGGAATGTGTGGGCCACTCACGCCTGCGTTC
CCACCGATCCTAATCCTCAAGAGATCGTGCTGGAAAACGTGACCGAGAA
CTTCAACATGTGGAAGAACAACATGGTCGAGCAGATGCACACCGACATC
ATCAGCCTGTGGGACCAGAGCCTGAAGCCTTGCGTGAAGCTGACCCCTC
TGTGCGTGACCCTGAACTGCACCAACGTGAACGTGACCAACACCACCAA
CAACACCGAGGAAAAGGGCGAGATCAAGAACTGCAGCTTCAACATCACC
ACCGAGCTGCGGGACAAGAAAAAGAAGGTGTACGCCCTGTTCTACCGGC
TGGACGTGGTGCCCATCGACGATAACAACAACAACTCCAGCAATTACCG
GCTGATCAACTGCAACACCAGCGCCATCACTCAGGCCTGTCCTAAGGTG
TCCTTCGAGCCCATTCCTATCCACTACTGTGCCCCTGCCGGCTTCGCCA
TCCTGAAGTGCAACGACAAGAAGTTCAACGGCACAGGCCCCTGCAAGAA
CGTGTCCACCGTGCAGTGTACCCACGGCATCAAGCCAGTGGTGTCTACC
CAGCTGCTGCTGAATGGCTCTCTGGCCGAGGAAGAGATCATCATCAGAA
GCGAGAACATCACGAACAACGCCAAGACCATCATCGTGCAGCTGAACGA
GAGCGTGGAAATCAATTGCACCCGGCCTAACAACAATACCCGGAAGTCC
ATCAGAATCGGCCCTGGCCAGTGGTTTTATGCCACCGGCGATATTATCG
GCGACATCAGACAGGCCCACTGTAACATCAGCGGCACCAAGTGGAACAA
GACCCTGCAGCAGGTCGTGAAGAAGCTGAGAGAGCACTTCAACAACAAG
ACGATCATCTTCAACCCCAGCTCTGGCGGCGACCTGGAAATCACCACAC
ACAGCTTCAATTGTGGCGGCGAGTTCTTCTACTGCAATACCTCCGGCCT
GTTCAACAGCACCTGGATCGGCAATGGCACCAAGAACAACAACAACACC
AACGACACCATCACACTGCCCTGCCGGATCAAGCAGATCATCAATATGT
GGCAGCGCGTGGGCCAGCCTATGTACGCTCCTCCAATCCAGGGCAAGAT
CAGATGCGTGTCCAATATCACCGGCCTGCTGCTCACAAGAGATGGCGGA
AACAACAACACGAATGAGACAGAGACATTCAGACCCGGCGGAGGCGACA
TGAGAGACAATTGGAGAAGCGAGCTGTACAAGTACAAGGTGGTCAAGAT
CGAGCCCCTGGGCGTCGCACCTACACGGTGCAAAAGAAGAGTGGTCGAA
GGCGGCGGAGGAAGCGGAGGCGGAGGATCTGCTGTTGGAATCGGAGCCG
TGTTCCTGGGCTTTCTGGGAGCCGCTGGATCTACAATGGGCGCTGCCAG
CATGACCCTGACAGTGCAGGCTAGAAATCTGCTGTCTGGCGGCAGCGGC
TCTGGCTCAGGATCTACAGTGTGGGGAATCAAGCAGCTGCAGGCCAGAG
TGCTGGCCGTCGAAAGATACCTGAGAGATCAGCAGCTCCTCGGCATCTG
GGGCTGTTCTGGCAAGCTGATCTGCTGCACCAATGTGCCCTGGAACAGC
TCCTGGTCCAACAAGAGCCAGGACGAGATCTGGGACAACATGACCTGGA
TGGAATGGGACAAAGAGATTAACAACTATACGGACATCATCTACAGCCT
GATCGAGGAAAGCCAGAACCAGCAAGAGAAGAACGAGCAGGACCTGCTG
GCCCTGGATAAGTGGGCTTCCCTGTGGAATTGGTTCGACATCACCAACT
GGCTGTGGTACATCAAGGTGCTGAGCATCATTGCCATCTGCCTGGGCAG
CCTGGGCCTGATCCTGATCATTCTGCTGAGCGTGGTCGTGTGGAAACTG
CTGACAATCGTGGTGGCCAACCGGAACCGGATGGAAAACTTCGTGTACC
ACAAGCGGCGCAGAAGGCGGAGAGGATCTGGCGAAGGCAGAGGCTCTCT
GCTGACATGTGGCGACGTGGAAGAGAACCCTGGACCTATGGGATGCGTG
CAGTGCAAGGACAAAGAACCCAGCATCAGCATCCCCGCCGATCCTACAA
ACCCCAGACAGAGCATCAAGGCCTTTCCAATCGTGATCAACAGCGACGG
CGGCGAGAAGGGCAGACTGGTTAAGCAGCTGAGAACCACCTACCTGAAC
GACCTGGACACCCACGAGCCTCTGGTCACCTTCGTGAACACCTACGGCT
TCATCTACGAACAGGACCGGGGCAACACAATCGTCGGCGAAGATCAGCT
GGGCAAGAAACGGGAAGCCGTGACAGCCGCCATGGTCACACTTGGCTGT
GGCCCTAATCTGCCTAGCCTGGGCAATGTGCTTGGCCAGCTGAGCGAGT
TCCAAGTGATTGTGCGCAAGACCAGCAGCAAGGCCGAAGAGATGGTGTT
CGAGATCGTGAAGTACCCCAGAATCTTCCGGGGCCACACACTGATCCAG
AAAGGCCTCGTGTGTGTGTCCGCCGAGAAGTTCGTGAAGTCTCCCGGCA
AGGTGCAGAGCGGCATGGACTACCTGTTCATCCCCACCTTTCTGAGCGT
GACCTACTGTCCTGCCGCCATCAAGTTCCAGGTGCCAGGACCTATGCTG
AAGATGCGGAGCAGATACACCCAGTCTCTGCAGCTGGAACTGATGATCA
GAATCCTGTGCAAGCCCGACAGTCCCCTGATGAAGGTGCACATCCCCGA
CAAAGAAGGCAGGGGCTGTCTCGTGTCTGTGTGGCTGCACGTGTGCAAC
ATCTTCAAGAGCGGCAACAAGAACGGCAGCGAGTGGCAAGAGTACTGGA
TGCGGAAGTGCGCCAACATGCAGCTCGAAGTGTCTATCGCCGACATGTG
GGGCCCTACCATCATCATCCACGCCAGAGGACACATCCCCAAGAGCGCC
AAGCTGTTCTTTGGCAAAGGCGGCTGGTCCTGCCATCCTCTGCATGAGG
TTGTGCCCAGCGTGACCAAGACACTTTGGAGCGTGGGCTGCGAGATCAC
CAAGGCCAAGGCCATTATCCAAGAGAGCAGCATCTCCCTGCTGGTGGAA
ACCACAGACATCATTAGCCCCAAAGTGAAGATCTCCAGCAAGCACAGAA
GATTCGGCAAGAGCAACTGGGGCCTGTTTAAAAAGACCAAGAGCCTGCC
TAACCTGACCGAGCTGGAATAG

Hence, preferably the fusion protein of the seventh may be encoded by a nucleic acid comprising a nucleotide sequence substantially as set out in SEQ ID NO: 25, or a variant or fragment thereof. Preferably, the antigen is a viral antigen.

In an eighth aspect, there is provided a nucleic acid sequence comprising a nucleic acid encoding the fusion protein of the seventh aspect.

The nucleic acid sequence may be a DNA, RNA or DNA/RNA hybrid sequence. Preferably the nucleotide sequence is a DNA or RNA sequence. In one embodiment, the nucleic acid sequence is a DNA sequence. In another embodiment the nucleic acid sequence is an RNA sequence. The RNA sequence may be an mRNA sequence or a self-replicating RNA sequence.

Any of the nucleic acids described herein may be isolated. The nucleic acids described herein may be purified, preferably to at least 90%, 91%, 92%, 93%, 94%, 95%, 96%, 97%, 98%, 99% or 100% purity.

In a ninth aspect of the invention, there is provided a VLP comprising a fusion protein according to the first aspect and a fusion protein according to the third aspect, wherein the antigen of the fusion protein of the first aspect is displayed on the surface of the VLP.

It will be appreciated that the VLP of the ninth aspect displaying the antigen is referred to as being pseudotyped or decorated. Preferably, the pseudotyped VLP comprises or displays at least 50 antigen molecules on its outer surface, more preferably at least 100, 200, or 500 antigen molecules. Even more preferably, the pseudotyped VLP comprises or displays at least 1000, 1500, 2000, 2500 3000, 3500, 4000, 4500 or 5000 antigen molecules. Preferably, the antigen is a viral antigen and the skilled person would understand that the display of viral proteins (i.e. antigen), activates the B cells when engaged by a B cell receptor (BCR), and leads to in the production of specific antibodies to the viral protein.

The average diameter of the VLP of the ninth aspect may be between 30 nm and 1000 nm, 40 um and 900 nm, 50 nm and 800 nm, 60 nm and 700 nm, 70 nm and 600 nm, 80 nm and 500 nm, 90 nm and 400 nm, 100 nm and 300 nm.

Preferably, the average diameter is between 30 nm and 1000 nm, 900 nm, 800 nm, 700 nm, 600 nm, 500 nm, 400 nm, 300 nm, 200 nm or 100 nm. Preferably, the average diameter is between 40 nm and 1000 nm, 900 nm, 800 nm, 700 nm, 600 nm, 500 nm, 400 nm, 300 nm, 200 nm or 100 nm. Preferably, the average diameter is between 50 nm and 1000 nm, 900 nm, 800 nm, 700 nm, 600 nm, 500 nm, 400 nm, 300 nm, 200 nm or 100 nm. Preferably, the average diameter is between 60 nm and 1000 nm, 900 nm, 800 nm, 700 nm, 600 nm, 500 nm, 400 nm, 300 nm, 200 nm or 100 nm. Preferably, the average diameter is between 70 nm and 1000 nm, 900 nm, 800 nm, 700 nm, 600 nm, 500 nm, 400 nm, 300 nm, 200 nm or 100 nm. Preferably, the average diameter is between 80 nm and 1000 nm, 900 nm, 800 nm, 700 nm, 600 nm, 500 nm, 400 nm, 300 nm, 200 nm or 100 nm. Preferably, the average diameter is between 90 nm and 1000 nm, 900 nm, 800 nm, 700 nm, 600 nm, 500 nm, 400 nm, 300 nm, 200 nm or 100 nm. Preferably, the average diameter is between 100 nm and 1000 nm, 900 nm, 800 nm, 700 nm, 600 nm, 500 nm, 400 nm, 300 nm, 200 nm.

A skilled person would appreciate that the diameter of a VLP may be determined using: Nanoparticle Tracking Analysis, dynamic light scattering or tunable resistive pulse sensing (TRPS), techniques that allows high-throughput single particle measurements as colloids and/or biomolecular analytes.

In a tenth aspect, there is provided a method of producing a pseudotyped virus like particle (VLP), the method comprising contacting a fusion protein of the first aspect with a fusion protein of the third aspect under conditions such that the antigen of the fusion protein of the first aspect is displayed on the surface of the VLP, thereby forming a pseudotyped virus like particle.

The contacting may be performed in vitro or ex-vivo.

In one embodiment, the contacting of a fusion protein of the first aspect with the fusion protein of the third aspect comprises contacting a fusion protein of the first aspect with a VLP that has been formed from the fusion protein of the third aspect, wherein that the fusion protein of the first aspect interacts with the VLP such that in the antigen of the fusion protein of the first being displayed on the outer surface of the VLP. Preferably, the fusion proteins interact with each other to result in pseudotyping the VLP.

In one embodiment, contacting of a fusion protein of the first aspect with the fusion protein of the third aspect comprises co-expression of the fusion protein of the first aspect with the fusion protein of the third aspect in a host cell, such that when expressed in the host cell, the fusion protein of the first aspect interacts with fusion protein of the third aspect such that the antigen is directed to the outer surface of the VLP formed by the fusion protein of the third aspect, thus forming a pseudotyped virus like particle. The VLPs may be secreted in the supernatant. The supernatants containing the VLPs may be harvested, and VLPs purified from cell according to standard processes for virus or VLP purification, which would be known to those skilled in the art.

The host cell may be a eukaryotic or prokaryotic host cell. Preferably, the host cell is a eukaryotic host cell. More preferably, the host cell is a mammalian host cell such as Human embryonic kidney 293 cells or Chinese hamster ovary (CHO) cells. Co-expression may be performed by transient transfection or through the establishment of stable cells lines.

In an eleventh aspect, there is provided use of the fusion protein of the first aspect for antigen display on a virus like particle.

Preferably, the virus like particle is as defined in the third aspect.

Preferably, antigen display is as defined in the first aspect.

In a twelfth aspect, there is provided an expression cassette comprising a nucleic acid according to the second, fourth and/or eighth aspect, or encoding the fusion protein of the first, third and/or seventh aspect.

The nucleic acid sequences of the invention are preferably harboured in a recombinant vector, for example a recombinant vector for delivery into a host cell of interest.

Accordingly, in a thirteenth aspect, there is provided a recombinant vector comprising the expression cassette according to the twelfth aspect.

The vector may for example be a plasmid, cosmid or phage and/or be a viral vector.

Such recombinant vectors are highly useful in the delivery systems of the invention for transforming cells with the nucleotide sequences. The nucleotide sequences may preferably be a DNA sequence.

Preferably the vector is a viral vector. The viral vector may be selected from the group consisting of an adeno associated vector (AAV), lentiviral vector, adenoviral vector and retroviral vector. Preferably, the vector is an AAV vector.

Recombinant vectors may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required. Tissue specific promoter/enhancer elements may be used to regulate expression of the nucleic acid in specific cell types, for example, epithelial cells. The promoter may be constitutive or inducible.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene. The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into cells of a host subject (e.g. a eukaryotic or prokaryotic cell) by transfection, infection, electroporation, microinjection, cell fusion, protoplast fusion or ballistic bombardment. Alternatively, vectors of the invention may be introduced directly into a host cell using a particle gun.

The nucleic acid molecule may (but not necessarily) be one, which becomes incorporated in the DNA of cells of the subject being treated. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells in the subject being treated. When this is the case, regulation of expression may be less important because expression of the DNA molecule will stop when the transformed cells die or stop expressing the protein (ideally when the required therapeutic effect has been achieved).

Alternatively, the delivery system may provide the nucleic acid molecule to the subject without it being incorporated in a vector. For instance, the nucleic acid molecule may be incorporated within a liposome or virus particle. Alternatively a “naked” nucleic acid molecule may be inserted into a subject's cells by a suitable means e.g. direct endocytotic uptake.

The nucleic acid molecule may be transferred to the cells of a subject to be treated by transfection, infection, microinjection, cell fusion, protoplast fusion or ballistic bombardment. For example, transfer may be by ballistic transfection with coated gold particles, liposomes containing the nucleic acid molecule, viral vectors (e.g. adenovirus) and means of providing direct nucleic acid uptake (e.g. endocytosis) by application of the nucleic acid molecule directly.

In a fourteenth aspect, there is provided a host cell comprising the nucleic acid sequence encoding the fusion proteins of the first, third or seventh aspect, the nucleic acid sequence of the second, fourth or eighth aspect, the expression cassette of the twelfth aspect, or the recombinant vector of the thirteenth aspect.

The host cell may be a eukaryotic or prokaryotic host cell. Preferably, the host cell is a eukaryotic host cell. More preferably, the host cell is a mammalian host cell.

In a fifteenth aspect, there is provided a pharmaceutical composition comprising the fusion protein of the first, third or seventh aspect; the nucleic acid sequence of the second, fourth or eighth aspect; the VLP of the fifth or ninth aspect; the expression cassette of the twelfth aspect; the vector of the thirteenth aspect; or the host cell of the fourteenth aspect, and a pharmaceutically acceptable vehicle.

In another embodiment, the nucleic acid sequence of the second and fourth aspects are present in the pharmaceutical composition, and are in the same expression cassette or vector.

In another embodiment, the nucleic acid sequence of the second and fourth aspects are present in the pharmaceutical composition, and are in a different expression cassette or vector.

In a sixteenth aspect, there is provided a process for making the pharmaceutical composition according to the fifteenth aspect, the method comprising contacting the fusion protein of the first and/or third aspect; the fusion protein of the seventh aspect; the nucleic acid sequence of the second, fourth or eighth aspects; the VLP of the fifth or ninth aspect; the expression cassette of the twelfth aspect; the vector of the thirteenth aspect; or the host cell of the fourteenth aspect, with a pharmaceutically acceptable vehicle.

It will be appreciated that the fusion proteins and VLPs of the invention could be used in therapy and diagnosis.

Hence, in a seventeenth aspect, there is provided the fusion protein of the first, third or seventh aspect; the nucleic acid sequence of the second, fourth or eighth aspect; the VLP of the fifth or ninth aspect; the expression cassette of the twelfth aspect; the vector of the thirteenth aspect; the host cell of the fourteenth aspect; or the pharmaceutical composition of the fifteenth aspect, for use in therapy or diagnosis.

In an eighteenth aspect, there is provided the fusion protein of first, third or seventh aspect; the nucleic acid sequence of the second, fourth or eighth aspect; the VLP of the fifth or ninth aspect; the expression cassette of the twelfth aspect; the vector of the thirteenth aspect; the host cell of the fourteenth aspect; or the pharmaceutical composition of the fifteenth aspect, for use in the prevention, amelioration or treatment of a viral infection.

In a nineteenth aspect of the invention, there is provided a method of treating a viral infection, the method comprising administering, to a subject in need thereof, a therapeutically effective amount of the fusion protein of the first, third or seventh aspect; the nucleic acid sequence of the second, fourth or eighth aspect; the VLP of the fifth or ninth aspect; the expression cassette of the twelfth aspect; the vector of the thirteenth aspect; the host cell of the fourteenth aspect; or the pharmaceutical composition of the fifteenth aspect.

Preferably, the viral infection to be prevented, ameliorated or treated is a viral infection selected from the group consisting of: HIV, Ebola virus, Marburg virus, Influenza, Measles virus, Mumps virus, Respiratory syncytial virus, Rinderpest virus, Nipha virus, Lassa virus, SARS corona virus, Herpes simplex virus 1, Epstein-Barr Virus, Dengue virus, Hepatitis C virus, Yellow fever virus, Zika virus, Rift Valley fever, or Rubella virus infection. Preferably, the viral infection is a HIV infection.

The fusion proteins described herein provide an effective means of vaccinating a subject against a viral infection.

Accordingly, in a twentieth aspect, there is provided a vaccine comprising the fusion protein of the first, third or seventh aspect; the nucleic acid sequence of the second, fourth or eighth aspect; the VLP of the fifth or ninth aspect; the expression cassette of the twelfth aspect; the vector of the thirteenth aspect; the host cell of the fourteenth aspect; or the pharmaceutical composition of the fifteenth aspect.

The vaccine may be a protein vaccine, a DNA vaccine or an RNA vaccine. Preferably, the vaccine comprises a suitable adjuvant.

In one embodiment, the vaccine may comprise fusion protein of the first, third or seventh aspect, the VLP of the fifth or ninth aspect. In this embodiment, the vaccine is preferably delivered to the bloodstream by injection.

In one embodiment, the vaccine may comprise a nucleic acid sequence of the second, fourth or eighth aspects; the expression cassette of the twelfth aspect; or the vector of the thirteenth aspect. In this embodiment, the vaccine is preferably delivered to the skin or muscle by injection.

When administered as a purified VLP the composition may be delivered with an adjuvant to enhance the magnitude and kinetics of induced immune response. Suitable adjuvants include those selected from the group consisting of: Aluminium salts (Alum), Lipid A analogues (e.g. MPLA, RC529, GLA, E6020), AS05 (MPL, aluminium salt), Emulsions (e.g. MF59, AS03, GLA-SE), imidazoquinolines (e.g. imiquimod, R848), CpG ODNs, Saponins (e.g. Q512), AS)1 (MPL, Q521, liposomes), AS02 (MPL, Q521, emulsion), AS15 (MPL, Q521, GpG, liposomes), CAF01 (TDB, cationic liposomes), ISCOMS (saponin, phospholipids), dsRNA analogues (e.g. Poly-IC), Flagellin, C-type lectins (e.g. TDB), CD1d ligands (e.g. alpha-galactosylceramide), IC31 (CpG, cationic peptides) and recombinant cytokines (e.g. IL-12, GM-CSF, type 1 interferons). In some embodiments cytokines may be encoded within the RNA sequence of the invention.

In a twenty-first aspect, there is provided the fusion protein of the first, third or seventh aspect; the nucleic acid sequence of the second, fourth or eighth aspect; the VLP of the fifth or ninth aspect; the expression cassette of the twelfth aspect; the vector of the thirteenth aspect; the host cell of the fourteenth aspect; or the pharmaceutical composition of the fifteenth aspect, for use in stimulating an immune response in a subject.

For example, the immune response may be stimulated against a protozoa, bacteria, virus, cancer, or a protein associated with neurodegenerative disorder as per the antigens defined in the first aspect.

It will be appreciated that the fusion protein of the first, third or seventh aspect; the nucleic acid sequence of the second, fourth or eighth aspect; the VLP of the fifth or ninth aspect; the expression cassette of the twelfth aspect; the vector of the thirteenth aspect; the host cell of the fourteenth aspect; or the pharmaceutical composition of the fifteenth aspect (herein known as the active agents) may be used in a medicament, which may be used as a monotherapy (i.e. use of the active agent), for treating, ameliorating, or preventing viral infection. Alternatively, the active agents according to the invention may be used as an adjunct to, or in combination with, known therapies for treating, ameliorating, or preventing a viral infection. For example, suitable antivirals may include: Entry inhibitors, for example the anti-HW drug Maraviroc; uncoating inhibitors such as Amantadine and Rimantadine to combat influenza; reverse transcription inhibitors such as antiviral, aciclovir, a nucleoside analogue, against herpesvirus infections and the wide range of nucleoside analogues and non-nucleoside analogues used against HIV that would be known to those skilled in the art; integrase inhibitors, such as raltegravir, dolutegravir, or elvitegravir; or protease inhibitors such as lopinavir, nelfinavir, ritonavir or saquinavir against HIV.

The fusion protein, VLP, nucleic acid sequence, expression cassette, recombinant vector or the pharmaceutical composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The fusion protein, VLP, nucleic acid sequence, expression cassette, recombinant vector or the pharmaceutical composition of the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the genetic construct or the recombinant vector is required and which would normally require frequent administration (e.g. at least daily injection).

In a preferred embodiment, however, medicaments according to the invention may be administered to a subject by injection into the blood stream, muscle, skin or directly into a site requiring treatment. Injections may be intravenous (bolus or infusion) or subcutaneous (bolus or infusion), or intradermal (bolus or infusion).

It will be appreciated that the amount of fusion protein, VLP, nucleic acid sequence, expression cassette, recombinant vector or the pharmaceutical composition that is required is determined by its biological activity and bioavailability, which in turn depends on the mode of administration, the physiochemical properties of the fusion protein, VLP, nucleic acid sequence, expression cassette, recombinant vector or the pharmaceutical composition and whether it is being used as a monotherapy or in a combined therapy. The frequency of administration will also be influenced by the half-life of the active agent within the subject being treated. Optimal dosages to be administered may be determined by those skilled in the art, and will vary with the particular the fusion protein, VLP, nucleic acid sequence, expression cassette, recombinant vector or the pharmaceutical composition in use, the strength of the pharmaceutical composition, the mode of administration, and the type and advancement of the viral infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of the fusion protein, VLP, nucleic acid sequence, expression cassette, recombinant vector or the pharmaceutical composition of the invention may be used for treating, ameliorating, or preventing a viral infection, depending upon the active agent used.

The fusion protein, VLP, nucleic acid sequence, expression cassette, recombinant vector or the pharmaceutical composition may be administered before, during or after onset of the viral infection. Daily doses may be given as a single administration (e.g. a single daily injection or inhalation of a nasal spray). Alternatively, the fusion protein, VLP, nucleic acid sequence, expression cassette, recombinant vector or the pharmaceutical composition may require administration twice or more times during a day. As an example, the fusion protein, VLP, nucleic acid sequence, expression cassette, recombinant vector or the pharmaceutical composition may be administered as two (or more depending upon the severity of the viral infection being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the fusion protein, VLP, nucleic acid sequence, expression cassette, recombinant vector or the pharmaceutical composition according to the invention to a patient without the need to administer repeated doses.

Known procedures, such as those conventionally employed by the pharmaceutical industry (e.g. in vivo experimentation, clinical trials, etc.), may be used to form specific formulations of the fusion protein, VLP, nucleic acid sequence, expression cassette, recombinant vector according to the invention and precise therapeutic regimes (such as daily doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the fusion protein, VLP, nucleic acid sequence, expression cassette, recombinant vector or the pharmaceutical composition is any amount which, when administered to a subject, is the amount of the aforementioned that is needed to ameliorate, prevent or treat the viral infection.

For example, the fusion protein, VLP, nucleic acid sequence, expression cassette, recombinant vector and the pharmaceutical composition of the invention may be used may be from about 0.01 mg to about 800 mg, and preferably from about 0.01 mg to about 500 mg. It is preferred that the amount of the fusion protein, VLP, nucleic acid sequence, expression cassette, recombinant vector or the pharmaceutical composition is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. fusion protein, VLP, nucleic acid sequence, expression cassette or recombinant vector of the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The fusion protein, VLP, nucleic acid sequence, expression cassette or recombinant vector according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The fusion protein, VLP, nucleic acid sequence, expression cassette or recombinant vector of the invention may be prepared as a sterile solid composition that may be dissolved or suspended at the time of administration using sterile water, saline, or other appropriate sterile injectable medium.

The fusion protein, VLP, nucleic acid sequence, expression cassette, recombinant vector and the pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The fusion protein, VLP, nucleic acid sequence, expression cassette, recombinant vector of the invention and the pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-34 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequence shown in, for example, SEQ ID No:3.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

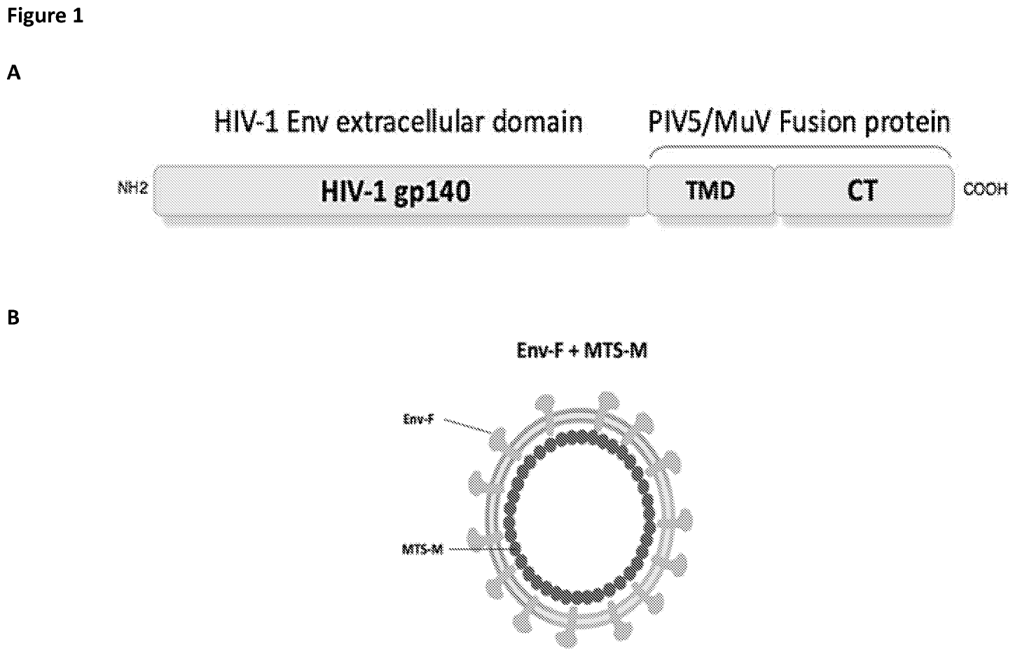

FIG. 1 shows the MTS-M, HIV Env-F chimera and Virus-Like Particles (VLPs) structure. (A) Diagram representing the Env-F chimera protein using the extracellular domain of HIV-1 Envelope (Env) and the transmembrane domain (TMD) and cytoplasmic tail (CT) of Mumps or PIV5 for pseudotyped VLPs. (B) Schematic structure of the VLPs using 3 proteins on the left sketch and 2 on the right sketch. M, NP and MTS-M are derived from PIV5 or Mumps.

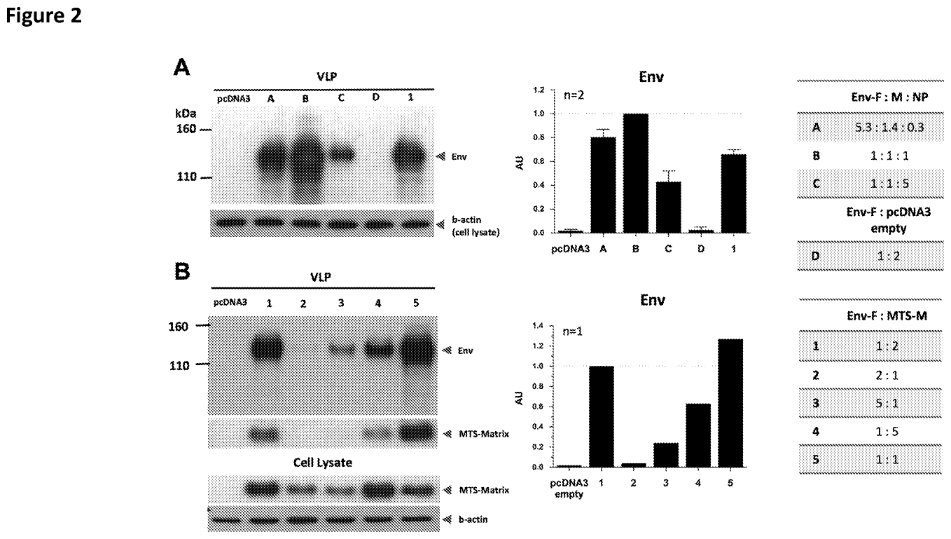

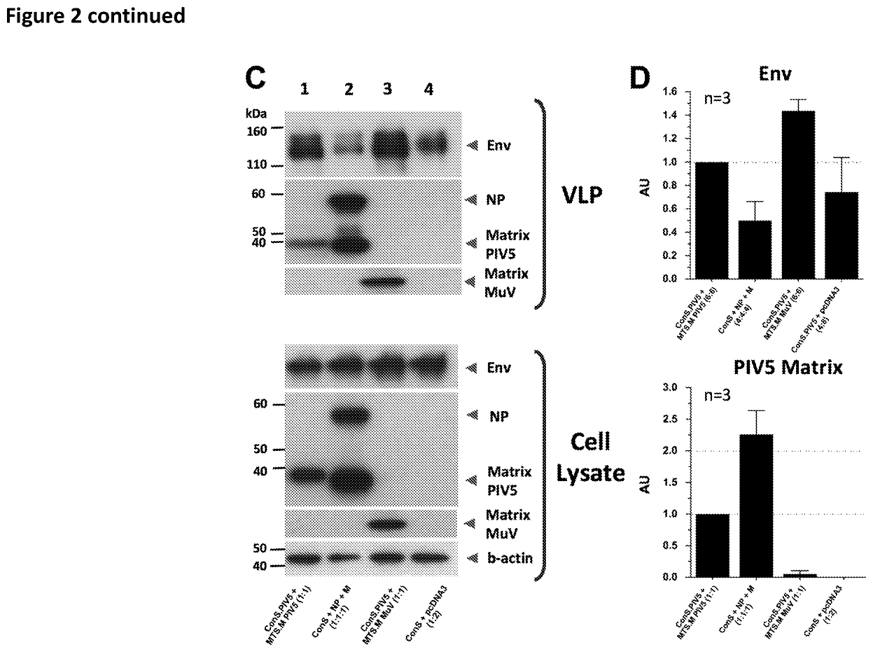

FIG. 2 shows that MTS-Matrix+Env-F chimera is sufficient to produce VLPs. (A) Western blot analyses of Mumps VLPs produced in HEK293T.17 cells using different ratios (w:w) of plasmids expressing (see table on the left): Env-F 1086C-MuV; M, matrix; NP; nucleoprotein; pcDNA3 empty. Densitometry analyses was performed and results for Env specific signal is reported in the middle panel with n=2. AU: arbitrary unit. (B) Same as in (A) using different ratios (w:w) of MTS-M (membrane targeting signal-matrix) MuV and Env-F 1086C-MuV (n=1) (C) Western blot analysis of PIV5 and MuV pseudotyped VLPs. ConSOSLUFO.P1V5 (ConS.PIV5) was tested in combination with the matching M+NP or MTS-M from PIV5 as well as with MTS-M MuV and pcDNA3 empty. (D) Densitometry analysis of the Env specific and PIV5 matrix specific signal from the VLP western blot in (C), with n=3. Error bars represent means+/−SEM.

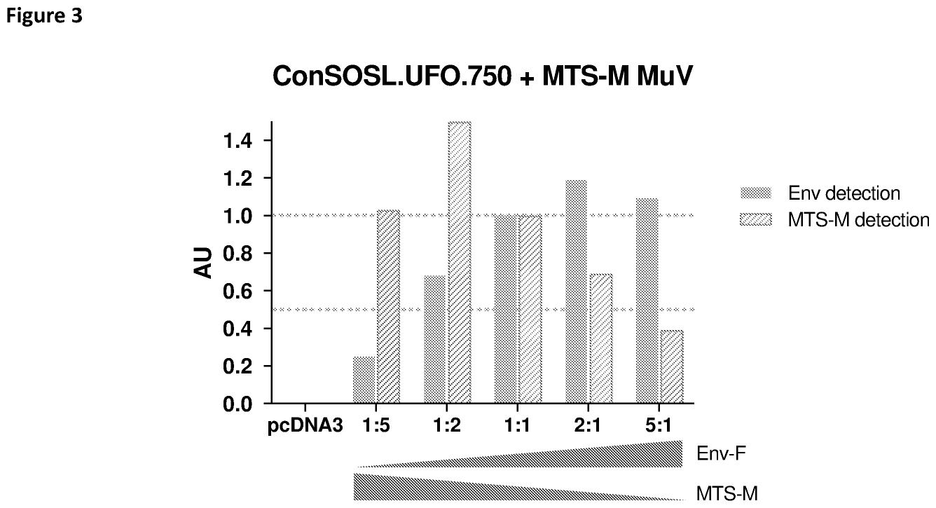

FIG. 3 shows that MuV MTS-Matrix can produce VLPs with a native HIV-1 truncated Env. VLPs were produced using different ratios (w:w) of plasmids Env (ConSOSLUFO.750) and MTS-M MuV. Western blots were analysed by densitometry and Env specific and MTS-M specific signal are plotted and normalized to the 1:1 ratio condition. n=1 experiment.

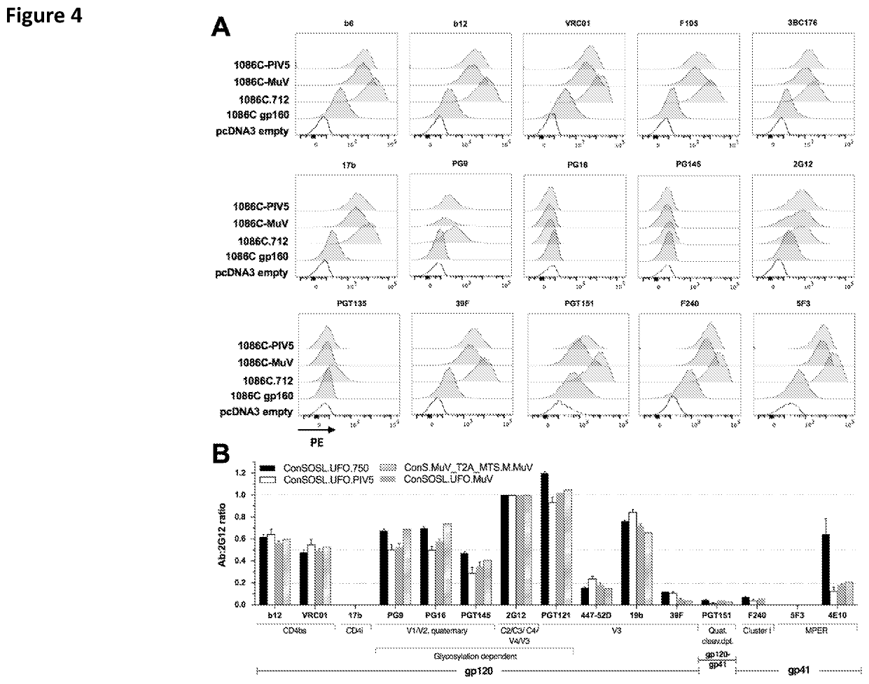

FIG. 4 shows that Env-F chimeras preserve the original HIV-1 Env ectodomain structure. (A) Flow cytometry analysis of HEK293T.17 cells transfected with Env-F chimera 1086C-PIV5 and 1086C-MuV or the matching wild type 1086C clade C HIV-1 Env truncated at position 712 (1086C.712) or full length Env (1086C gp160). Cells were stained with a panel of monoclonal antibodies (mAb) specific for different domains of Env and a negative control included (pcDNA3 empty). (B) Flow cytometry analysis of Env-F pseudotyped using a stabilized HIV-1 Env (ConSOSLUFO.750) that binds preferentially broadly neutralizing antibodies. The mean fluorescence intensity values were normalized to 2G12 mAb. mAb are found on the x-axis and organized by Env domain specificity. ConS.MuV_T2A_MTS.M.MuV co-expresses ConSOSL.UFO.MuV along with MTS-Matrix MuV from the same transcript where the two proteins are separated by a virally derived (T2A) self-cleavage peptide.

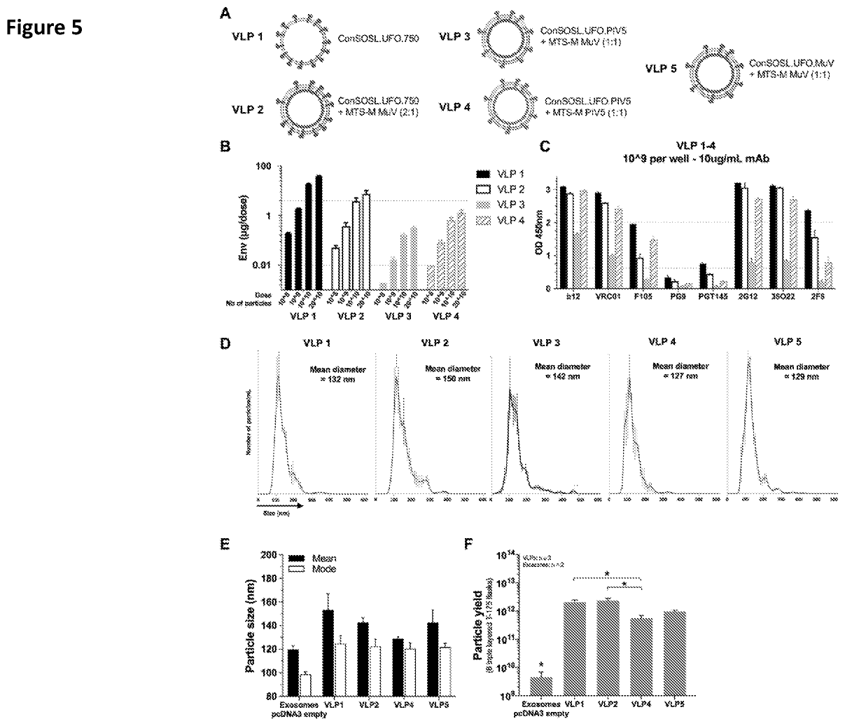

FIG. 5 shows Env quantification and antigenic profile of the Env-F on VLPs. (A) Diagram representing the different type of VLPs. The red Env represents ConSOSLUFO.750. The Env-F and MTS-M (inner plain circles) are purple for MuV and yellow for PIV5. The ratio (w:w) used for transfection of HEK293T.17 cells when two vectors are used is indicated in brackets. (B) Quantification of the Env on the surface of the VLPs1-4 by capture ELISA using GNL for capture and ConSOSL.UFO.664 gp140 Env protein as stantard. (C) Evaluation of the VLP Env antigenicity by GNL capture ELISA for VLP1-4. 10E9 VLPs for each type of VLPs were captured and monoclonal antibodies specific for Env used at 10 μg/mL. (D) The VLP size distribution and number were characterized on a Nanosight instrument using Nanoparticle Tracking Analysis. The mean diameter of each type of VLP is indicated for the measurement displayed. (E) VLP1, VLP2, VLP4 and VLP5, as well as exosomes (from pcDNA3 empty vector transfected cells), from 6 triple layered T-175 flasks of transfected HEK293T.17 cells were purified by ultracentrifugation through a 20% sucrose cushion and their size analyse on a Nanosight. Particle size mean (black bars) and mode (white bars) in nm are depicted. Error bars represent mean±SEM with n≥3 for the VLPs and n=2 for the exosomes. (F) The total yields of VLPs and exosomes from the same productions as in part (E) are plotted. Error bars represent mean±SEM with n≥3 for the VLPs and n=2 for the exosomes, Welch's t-test with *p<0.05.

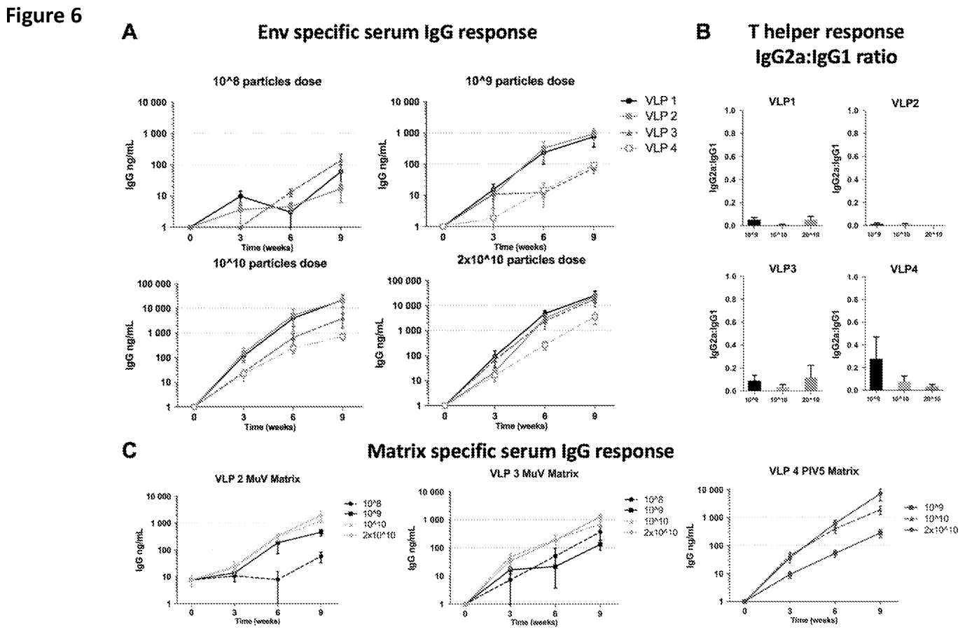

FIG. 6 shows that the VLPs are immunogenic without adjuvant and induce a Th2 type response in a mouse model. For subparts A-C, groups of n=5 mice were immunized intramuscularly with VLP1 to 4 three times at 3 weeks intervals with different VLP doses: 10E8, 10E9, 10E10 and 20E10 particles/injection. Serum samples were collected before each injection and before culling the animals. (A) Env specific IgG titers were determined by ELISA using the matching ConSOSL.UFO.664 gp140 Env protein. (B) Env specific IgG1 and IgG2a responses were assessed by ELISA and reported here as the IgG2a:IgG1 ratio as a surrogate for determining the type of T helper response. (C) The serum specific IgG responses against matrix PIV5 and matrix MuV were determined by ELISA for VLP2, VLP3 and VLP4 immunized animals. (D) Group of n=5 mice were immunized intramuscularly 3 times at 3 weeks intervals with VLP1 (10E8, 10E9 and 10E10 particles) plus AddaVax adjuvant (1:1, v:v ratio). Dashes lines: VLP1+AddaVax; Plain lines: VLP1 no adjuvant.

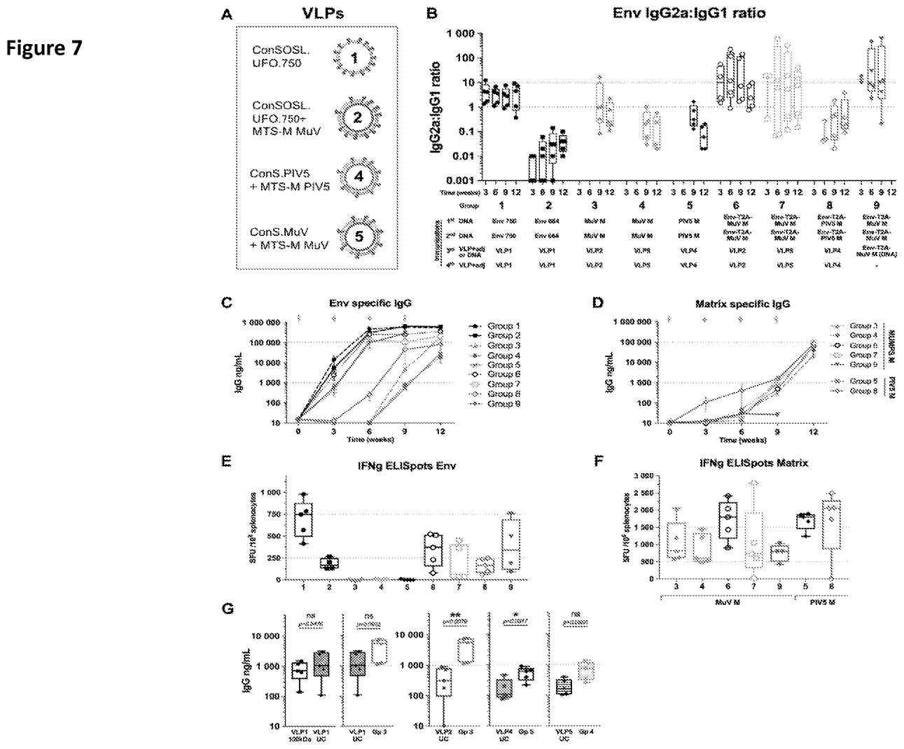

FIG. 7 shows that the IgG subtype response varies upon Env, MTS-Matrix or Env-T2A-MTS-Matrix DNA prime-VLP boost regimens and shows evidence of intrastructural help from MTS-Matrix DNA primed grouped. 9 groups of n=5 mice were primed twice at week 0 and week 3 with different DNA plasmids expressing Env (solube: Env 664; membrane-boud: Env 750), MTS-M MuV, MTS-M PIV5 or Env-F-T2A-MTS-M (PIV5 or MuV pseudotyped) and then received VLP boosts (10E10 particles+AddaVax adjuvant) at week 6 and 9, except one group receiving a total of 3 injections with ConS.MuV-T2A-MTS-M MuV (see immunization schedule in (B)). (A) Diagram of the different VLPs used with colour code as in FIG. 6A. (B) The IgG2a:IgG1 ratio for Env specific response is presented. It is used as a surrogate measure of the type T helper response induced by the different regimens throughout the immunization schedule. (C) Env specific IgG titers determined by ELISA. (D) Matrix specific IgG response for MuV (group 3, 4, 6, 7 and 9) and PIV5 (group 5 and 6). (E) IFNg specific Env ELISpost. Isolated splenocytes were stimulated with Env peptide pool for 16 hr. (F) IFNg specific matrix response. Isolated splenocytes were stimulated with either MuV matrix peptide pool or PIV5 matrix peptide pool for 16 hr. Grey arrows: immunization. (G) Comparison of the Env specific IgG response induced by VLP1 purified on a 100 kDa MWCO columns (VLP1 100 kDa) (10E10 particles+AddaVax) and VLP1 purified by ultracentrifugation (VLP1 UC) (10E10 particles+AddaVax) after 1 injection (left panel). The remaining 4 panels display the Env IgG titers of group 3 (Gp3), 4 (Gp4) and 5 (Gp5) following the 1st VLP injection which are compared to the Env specific IgG titers induced by 1 injection of VLP1 UC, VLP2 UC, VLP4 UC or VLP UC (10E10 particles+AddaVax). The same batch of VLPs UC were used for the DNA prime-VLP boost study and the VLP UC+AddaVax immunization

study. Box and whiskers, min to max. Mann-Whitney test with *p<0.05, **p<0.01, ns=non significant.

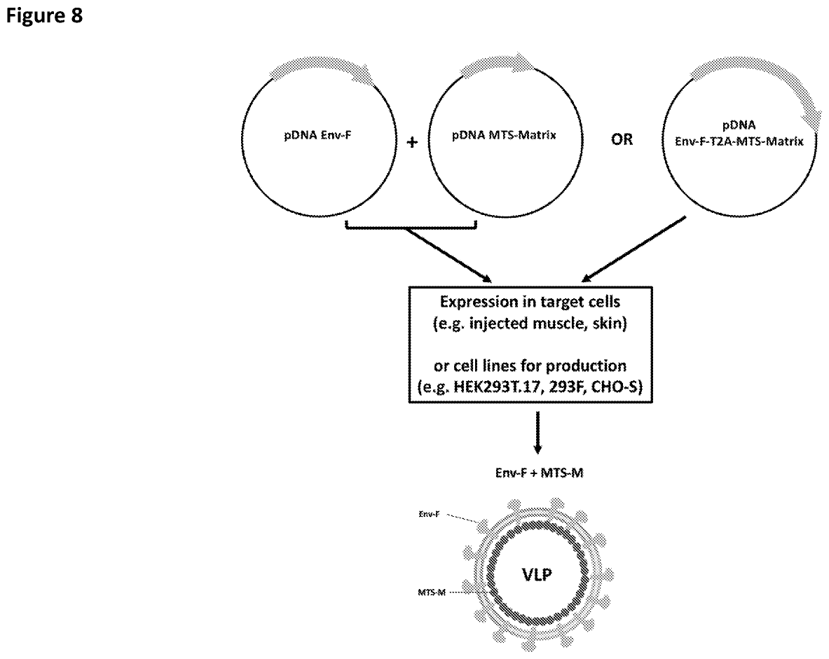

FIG. 8 shows that VLPs can be produced from by mixing separate expressing vectors coding for Env-F and MTS-Matrix and also that VLPs can be produced from a single pDNA coding for a gene bearing both Env-F and MTS-Matrix (pDNA Env-F-T2A-MTS-Matrix). The advantage of pDNA Env-F-T2A-MTS-Matrix that it produced one transcript bearing both Env-F and MTS-Matrix coding sequence which once translated is self-cleaved to free the MTS-Matrix from the Env-F. This ensures that if delivered as a nucleic acid vaccine, both proteins will be expressed in the same cells and never separately.

### EXAMPLE

### Materials and Methods

### Plasmid DNA Vectors

Plasmid DNA (pDNA) vectors expressing HIV-1 Env constructs, Env-F MuV chimera, Env-F PIV5 chimera, MuV matrix, PIV5 matrix, nucleoprotein (NP) MuV, NP PIV5, MTS-Matrix MuV and MTS-Matrix PIV5 were codon optimized for Homo sapiens expression and either created using published sequences or designed in silico, and cloned into pcDNA3.1(+) using GeneArt gene synthesis service (ThermoFisher Scientific). The different pDNA were transformed in chemically competent one shot TOP10 E. coli or DH5a bacteria (Invitrogen). 100 mL maxiprep cultures were grown in lysogeny broth (LB) media overnight at +37° C., 215 rpm. pDNA were then extracted using Plasmid Plus Maxi kits (Qiagen) following the manufacturer's instructions. pDNA were eluted from the Qiagen columns using molecular biology grade water HyClone (GE LifeSciences). The concentration was then measured on a NanoDrop instrument (Thermo Fisher Scientific) and pDNA stored at −20° C.

HIV-1 Monoclonal Antibodies (mAbs)

mAbs were obtained from their producers, purchased from commercial suppliers or produced in house. 2G12, PG9, PG16, b12, 447-52D, 5F3,4E10, 2F5 and F240 were acquired from Polymun Scientific (Austria); 17b was donated by James Robinson; 35O22 was obtained from the NIH AIDS Research and Reference Reagent Program; expression vectors for 39F, 19b, 3BC176, PGT121, PGT135, PGT145, F105 and b6 were obtained from the IAVI Neutralizing Ab Consortium and produced in house; expression vectors for VRC01 and PGTL51 were generated in house. In house mAbs were produced in HEK293T.17 cells (ATCC) and purified on HiTrap protein A HP column (GE LifeSciences) following the manufacturer's instructions.

### Flow Cytometry

Surface expression of the HIV-1 Env construct and the Env-F chimeras was evaluated in HEK293T.17 cells. Cells were seeded in complete medium 30h prior to overnight transfection using PEI with a 1:3 pDNA:PEI ratio (w:w) in DMEM (Sigma)+2 mM glutamine (GIBCO) without antibiotics and without fetal bovine serum. Following the overnight incubation, the transfection media was removed and replaced by 293 FreeStyle medium (GIBCO). 48h later, cells were rinsed with 1×PBS, dissociated with cell dissociation buffer (GIBCO) then washed with FACS buffer (2.5% FBS, 1 mM EDTA, 25 mM HEPES in 1×PBS) and pelleted at 600×g for 5 min. Cells were resupended in FACS buffer and counted in an haemocytometer using trypan blue. Cells were then filtered (70 um filter), stained with aqua viability dye (1:400) for 20 min at room temperature (RT) in the dark, washed twice with FACS buffer and transferred in U bottom 96-well plates for the rest of the staining procedure. 10 ug/mL in 100 mL FACS buffer of primary human IgG anti-Env mAbs were used to stain 1×10⁶ cells per well for 30 min at RT in the dark. Cells were then washed twice with 125 uL FACS buffer and 0.1 ug secondary F(ab′)2-goat anti-human IgG Fc PE conjugated (Invitrogen) per 10⁶ cells added to the cells in 100 uL FACS buffer. Cells were incubated in the dark for 20 min, washed twice, resuspended in 100 uL PBS and fixed with an additional 100 uL 3% paraformaldehyde (Polysciences) to reach a final 1.5%. Samples were acquired on a LSRFortessa FC (BD) using FACSDiva (BD) and data interpreted using FlowJo v.10.1 software (Treestar). Live cells were gated and data presented either as traces or reported as mAb:2G12 ratio in order to normalize the data using the mean fluorescence intensity (MFI) values of the live cells—2G12 mAb gives among the highest binding signal on our ConSOSL.UFO.750 HIV-Env design. A pcDNA3 empty vector transfected HEK293T.17 cells control was included in each experiment to allow subtraction of each mAb background (the majority of these mAb have no background).

### Virus-Like Particle (VLP) Production

HEK293T.17 cells were seeded 30h before transfection to reach 80-90% confluence for transfection. Cells were co-transfected with a combination of HIV-1 Env-F:Matrix:NP, Env-F:MTS-Matrix ratios for MuV and PIV5 VLP pseudotyping using PEI in a 1:3 DNA:PEI ratio (w:w) in DMEM+2 mM glutamine. The transfection media was left overnight on the cells at +37° C. and replaced after 16-17h by FreeStyle™ 293 medium (GIBCO). The supernatants containing the VLPs were harvested, cell debris pelleted at 2,000×g for 5 min and the supernatant filtered using 0.45 μm PES membrane filters (Corning).

For the first VLP productions (FIG. 2) were from T-75 flasks transfections. These VLPs were concentrated on 300 kDa MWCO Vivaspin (Sartorius) columns at 3000×g. Once the volume of the VLP supernatants reached under 1 mL, VLPs were washed with 5 mL of 1×PBS and further concentrated down to 100 μL. Protease inhibitor cocktail was added to the collected fractions and the VLPs stored at −80° C. Later, we used 100 kDa MWCO Vivaspin (Sartorius) columns to concentrate the VLPs and produced VLPs from T-75 flasks (FIG. 3) and from triple layered T-175 flasks which were used for the first animal studies (FIG. 6). Finally, to achieve higher purity we further purified the 100 kDa MWCO Vivaspin concentrated VLPs using 20% sucrose cushion ultracentrifugation. VLPs were ultracentrifuged in polycarbonate thick wall tubes (Beckman Coulter) using a Beckman Coulter type 70 Ti rotor at 90,000×g for 4h at +4° C. The supernatant and sucrose cushion were then removed carefully, the pellets washed with 5 mL of 1×PBS and then resuspended in 200-500 uL 1×PBS. Right after resuspending the VLPs, 5 uL of VLPs were used to analyse and count the particles on the Nanosight. VLPs were then aliquoted and stored at −80° C. These VLPs were used for the DNA prime-VLP boost experiment (FIG. 7).

### HIV-1 Env Soluble Trimer and MTS-Matrix HIS Tagged Proteins

ConSOSL.UFO.664 HIV-1 Env soluble trimers was produced in HEK293T.17 cells using polyethyleneimine (PEI) (Polysciences) for transfection with a 1:3 DNA:PEI (w:w) ratio. The supernatant of transfected cells was collected 48h post-transfection, spun to pellet cellular debris followed by filtration (0.22 um). The soluble HIV-1 Env trimers were concentrated and transferred in 1×phosphate buffer saline (PBS) using 100 kDa molecular weight cut-off (MWCO) Amicon ultrafiltration columns (Merck Millipore). Further purification steps include 2 rounds of size exclusion chromatography (SEC) on an NGC medium pressure liquid chromatography (MPLC) system (BioRad) using an Enrich SEC 650 column (BioRad) to isolate the protein from the trimer peak. Trimers were then aliquoted and stored at −80° C.

MTS-Matrix MuV HIS tagged and MTS-Matrix PIV5 HIS tagged proteins were produced using the same DNA:PEI ratio and transfection conditions as for ConSOSL.UFO.664. Cells debris were pelleted then the supernatants filtered (0.45 um). The supernatant were concentrated on 10 kDa MWCO Vivaspin columns (Sartorius) to reduce the volume input for the affinity column. 0.02% Tween20 (v:v) was added to the concentrated supernatants and the proteins purified on HisTrap HP 1 mL columns following the manufacturer's instructions and adding the 0.02% Tween20 (v:v) to the buffer to equilibrate the columns. Eluted fractions were concentrated and protein transferred in 1×PBS using 10 kDa MWCO Vivaspin columns at 4,000×g. Concentrations were determined using a NanoDrop instrument and proteins stored at −20° C.

### VLP Characterization

1. Nanoparticle Tracking Analysis

The VLP size was characterized using a NanoSight LM10 instrument (Malvern Instruments, UK) with a SCMOS camera. VLP samples were diluted in 1×PBS in order to reach the recommended concentration range of 10⁸ to 109 particles/mL for accurate measurements. The NanoSight NTA 3.0 software (Malvern Instruments, UK) was used to acquire the data using an automated syringe pump at speed 10. The slider shutter was set up at 470 and the slider gain at 350. 60 seconds videos were recorded 3 times for each samples and temperature recorded. Images were then analysed using a screen gain of 10, a detection threshold of 5 with the ‘blur’ function switched off.

2. Envelope Quantification by Capture ELISA

MaxiSorp high binding ELISA plates were coated overnight at +4° C. with Galanthus Nivalis Lectin (GNL) (Sigma) at 5 ug/mL in 100 uL per well in 1×PBS. Plates were then emptied, tap dry, wash 3 times with 200 uL 1×PBS. VLPs were diluted at 10⁷, 10⁸, 10⁹ and 10¹⁰ particles in 50 uL/well 0.5× casein buffer (½CB) (Thermo Scientific). VLPs were loaded onto the GNL coated plates as well as the ConSOSL.UFO.664 gp140 standard starting at 10 ug/mL (1/5 dilution series) in 50 uL/well ½CB. The plates were incubated at +37° C. for 1h, washed twice with 200 uL/well 1×PBS then mAb 2G12 was added at 2.5 ug/mL in 100 uL/well ½CB. Following 1h incubation at +37° C., plates were washed twice with 200 uL/well 1×PBS and the secondary goat anti-human IgG Fc biotinylated Ab (Southern Biotech) added onto the plate at 1:10,000 in 100 uL/well ½CB, 30 min at +37° C. Plates were then washed twice as per the previous wash and poly-HRP40 (Fitzgerald) diluted 1:10,000 in 100 uL/well ½CB added for 20 min at +37° C. Plates were then washed 3 times with 200 uL/well 1×PBS, tapped dry and developed using 50 uL/well TMB (KPL) and the reaction stopped using 50 uL/well Stop solution (Insight Biotechnologies, UK). The absorbance was read on a KC4 Spectrophotometer at 450 nm (BioTek).

3. VLP Env Antigenicity

10 ug/mL GNL was coated onto the MaxiSorp high binding ELISA plates. Following the same protocol as per the ‘Envelope quantification by capture ELISA’, 10⁹ particles per 50 uL/well ½CB were loaded onto the coated plate. Then different mAbs specific for Env extracellular domain were added at 10 ug/mL in 100 uL/well ½CB followed by secondary Ab, poly-HRP40 and development.

### Western Blotting

Samples were prepared in reducing conditions using SDS sample buffer (Invitrogen) plus DTT, boiled for 5 min at +95° C., briefly cooled at +4° C. then loaded onto polyacrylamide Novex Tris-Glycine gels (Invitrogen). Gels were run for 40 min at 225 V in SDS running buffer (Invitrogen). Proteins were then followed by transfer into nitrocellulose membranes (Invitrogen), 80 min at 10 V in transfer buffer containing 10% methanol. Membranes were blocked in blocking buffer (2% (w/v) Bovine Serum Albumin (BSA) (Sigma), 0.05% Tween20 (v/v) in 1×PBS) for 1h at room temperature on a tube roller. Membranes were then washed 3 times 10 min with 15 mL 1×PBS+0.05% Tween20 (v/v). Primary antibodies: mouse Ab b13 specific for HIV-1 Env (0.5 μg/mL), mouse anti-PIV5 NP (Ab 214) and/or Matrix (Ab 198) at 1:2,000 (provided by Richard Randall, St Adrews University, UK) or mouse anti-Matrix MuV (1:3,000) were then added in blocking buffer. The membranes were incubated with the primary antibodies overnight at +4° C. on a tube roller. The membranes were then washed 3 times and secondary Goat anti-Mouse IgG Fc biotinylated Ab (Southern Biotech) added at 1:15,000 in blocking buffer. After another washing step, the membranes were incubated with streptavidin-HRP 1:500 (R&D Systems), then washed 3 times, dried, WB Luminata® Classico (Merck Millipore) applied and finally developed on Amersham Hyperfilm ECL (GE LifeSciences). Densitometry analyses were carried out using Image Studio Lite software v5.2.5 and ploted using GraphPad Prism v7.0.

### Animals and Immunization

Animals were handled and procedures were performed in accordance with the terms of a project license granted under the UK Home Office Animals (Scientific Procedures) Act 1986.

For the first immunogenicity study using 100 kDa MWCO concentrated VLPs, 4 groups of n=5 female BALB/c mice were injected intramuscularly in the quadriceps 3 times at 3-week interval with 10⁸, 10⁹, 10¹⁰ or 2×10¹⁰ particles dose of VLP1, VLP2, VLP3 or VLP4 without adjuvant in 50 uL 1×PBS. For the second study, groups of n=5 female BALB/c mice were injected intramuscularly with 10⁸, 10⁹ and 10¹⁰ particles dose of VLP1 with AddaVax adjuvant (1:1 ratio, v:v) in 50 uL. For the DNA prime-VLP boost study, 9 groups of n=5 mice per group were immunized twice at 3-week interval with 20 ug of pDNA (cf. FIG. 7) in 50 uL 1×PBS followed by electroporation (EP) using 5-mm electrodes using an ECM 830 square-wave electroporation system (BTX) (3 pulses of 100 V each followed by 3 pulses of the opposite polarity with each pulse (P_{ON}) lasting 50 ms and an interpulse (P_{OFF}) interval of 50 ms). 3 weeks later, mice were boosted with 50 uL of 10¹⁰ particles dose of the different VLPs purified by ultracentrifugation plus AddaVax adjuvant (1:1 ratio, v:v) according to the different groups in FIG. 7, except group 9 who received a 3^{rd} DNA injection. The VLP boost was repeated 3 weeks later and mice sacrificed at week 12. For all animals, serum samples were collected at each immunization time point and spleens were collected and processed from the 3d immunization study.

### IFN-γ ELISpots

IFN-γ T cell response was assessed using the Mouse IFN-γ ELISpotPLUS kit (Mabtech) following the manufacturer's instructions. Briefly, anti-IFN-γ pre-coated plates were blocked with DMEM+10% FBS for at 2h, then cells were added at 2.5×10⁶ cells/well. The negative control wells had media only, Env specific well had HIV-1 Env ConSOSL.UFO.750 peptide pool (2.5 μg/mL), Matrix specific wells had either MTS-Matrix MuV or MTS-Matrix PIV5 peptide pool in 200 μL final volume per well. The positive control wells contained 5×10⁵ cells/well in 200 μL final volume per well with 5 μg/mL of ConA. Plates were incubated overnight at 5% CO₂, +37° C. incubator and developed as per the manufacturer's protocol. Once dried, plates were read using the AID ELISpot reader ELR03 and AID ELISpot READER software (Autoimmun Diagnostika GmbH, Ger).

### Antigen Specific ELISA

### HIV-1 Env Specific ELISA

MaxiSorp high binding plates where coated with ConSOSL.UFO.664 protein at 1 ug/mL, 100 uL/well in 1×PBS and 1:1,000 dilution of each of the capture goat anti-Kappa and anti-Lambda was used to coat the standard wells (Southern Biotech). After an overnight incubation at +4° C., plates were washed 4 times with 1×PBS-0.05% Tween20 then blocked with in ELISA buffer (1% BSA+0.05% Tween20 in 1×PBS) with 200 uL/well and incubated for 1h at +37° C. The plates were then washed as describe above, incubated with samples diluted 1:100, 1:1,000 and 1:10,000 in ELISA buffer and the standard IgG, IgG1 and IgG2a added to the standard wells (start at 1 10 ug/mL then 1:5 dilution series). Following a 1h incubation at +37° C., plates were washed, incubated with 1:2,000 secondary goat anti-IgG-HRP, IgG1-HRP or IgG2a-HRP (Southern Biotech) for 1h at +37° C. Finally, plates were washed and developed using 50 uL/well TMB substrate then stopped with 50 uL Stop solution and read on a spectrophotometer.

Matrix specific ELISAs

Plates were prepared and handle as above except that the antigens used to code the plates are MTS-Matrix MuV HIS tagged protein (1 ug/mL) or MTS-Matrix PIV5 HIS tagged protein (1 ug/mL).

### Example 1

To demonstrate the potential of generating VLPs pseudotyped with viral glycoproteins, the inventors used the external domain of HIV Env GP (the portion that is external to the viral membrane and the key target for antibody responses). They evaluated the potential of MuV and PIV5 TMD+CT Fusion chimera (Env-F) to retain HIV Env extracellular domain epitope properties (FIG. 1A). VLPs were produced in HEK293T.17 cells and characterized by western blot, ELISA and Nanoparticle Tracking Analysis (Figure B). In addition, VLPs can be produced when encoded in a DNA vector either with matrix and glycoprotein components delivered on separate plasmids or where the matrix and glycoprotein components are encoded in the same sequence separated by a T2A cleavage sequence (RRRRRRGSGEGRGSLLTCGDVEENPGP SEQ ID No:19).

The MuV TMD may be encoded by a nucleic acid having a nucleotide sequence comprising

SEQ ID No: 31
GTGCTGAGCATCATTGCCATCTGCCTGGGCAGCCTGGGCCTGATCCTGA
TCATTCTGCTGAGCGTGGTCGTG.

The MuV CT may be encoded by a nucleic acid having a nucleotide sequence comprising

SEQ ID No: 32
TGGAAACTGCTGACAATCGTGGTGGCCAACCGGAACCGGATGGAAAACT
TCGTGTACCACAAG.

The PIV5 TMD may be encoded by a nucleic acid having a nucleotide sequence comprising

SEQ ID No: 33
GCCATCATTGTGGCCGCTCTGGTGCTGAGCATCCTGTCCATCATCATCT
CCCTGCTGTTCTGCTGCTGGGCCTACGTG.

The PIV5 CT may be encoded by a nucleic acid having a nucleotide sequence comprising

SEQ ID No: 34
GCCACCAAAGAGATCAGACGGATCAACTTCAAGACCAACCACATCAACA
CCATCAGCTCCAGCGTGGACGACCTGATCAGATAC.

The inventors have shown that MuV/PIV5 MTS-M+Env-F is sufficient to produce VLPs (FIG. 2). In addition, the inventors have shown that MuV MTS-M can produce VLPs when co-expressed with the PIV5 Env-F chimera as well as with a HIV Env GP truncated at amino acid 750 (FIG. 3). Furthermore, using a panel of highly characterised anti-HIV Env antibodies the inventors found that the designed chimeric HIV Envs, using a wild type Env sequence (FIG. 4A) as well as a stabilized Env sequence developed by the inventors (FIG. 4B), preserve the quaternary structure and broadly neutralizing antibody (bNAb) binding profile of the matching HIV Env.

### Example 2

The inventors then quantified the amount of Env that was expressed on each of the VLP versions and analysed the antigen profile using a panel of well characterised anti-HIV Env antibodies (FIG. 5B-C). All the VLP versions expressed high levels of the recombinant HIV Env protein, much more than the levels observed on an HIV virion, demonstrating a clear advantage over the native virus. They also characterized the size distribution of the VLPs using Nanoparticle Tracking Analysis performed on a Nanosight instrument (FIG. 5D). For the 5 types of VLPs, >90% of the measured particles had a diameter between 90-250 nm, with a mean diameter of laying between 120-150 nm. These data demonstrate that particles are formed and have a similar size to the HIV virion.

### Example 3

The inventors next evaluated the immunogenicity in a mouse model of VLP1 to 4 and showed that the VLPs where immunogenic without the addition of a separate adjuvant from a dose of 10E9 particles (FIG. 6A, C). VLPs containing a MTS-M component showed mounted an antibody response against the matrix, response which appears to be one log lower than for Env. These VLPs induced a predominant Th2 response with a very low IgG2a:IgG1 ratio observed (FIG. 6B). In addition, they evaluated the potential of VLP1, which contains no matrix, in combination with a conventional adjuvant. The serum IgG response was efficiently boosted as expected (FIG. 6D). These data demonstrate the high immunogenic potential of the VLPs generated using the inventor's production method.

The inventors further tested VLP immunogenicity in the context of DNA prime-VLP boost regimens (FIG. 7). They found that priming with a DNA expressed membrane bound Env induced a Th1 response which was maintained following VLP1 boosts. Interestingly, priming with DNA expressed soluble Env induced a strong Th2 skew which was not reverted or balanced with a Th1 response, although the IgG2a:IgG1 increase slightly following VLP1 boosts. DNA MTS-M primed grouped showed either a balanced Th1/Th2 response or a Th2 skewed response with VLP2, 3 and 4 boosts. In addition, DNA prime with co-expressing vector ConSOSL.UFO.MuV-T2A-MTS-M MuV induced a Th1 skewed or Th1/Th2 balanced response which was maintained following the VLP2 and VLP5 immunization. In contrast, the PIV5 version of this DNA vector induced a Th2 skewed response. Strikingly, when no Env was used for DNA prime there was no Env IFN-gamma response observed (Group 3,4 and 5—FIG. 7F) whereas priming with DNA membrane-bound Env (group 1) gave a strong response in mark contrast with priming with DNA soluble Env (group 2). Interestingly, the inventors observed an increase of Env serum IgG titer for group 3 compared to VLP1+AddaVax after the first boost, and, without being bound to any particular theory, suggests intrastructural help from the MTS-M specific T helper cells induced by DNA priming which could potentially be recalled with VLP2 boosting (FIG. 7E). Moreover, VLP2 present less Env on its surface than VLP1 and the animals were injected with VLPs normalized to the number of VLPs and not Env.

### Discussion

The majority of commercialized vaccines generate protection against infectious viruses through the induction of protective antibodies. These protective antibodies typically target the viral glycoproteins arrayed on the surface of the viral particle (virus spikes). The correct display of these surface glycoproteins is thought to be advantageous to evoke the right type of protective antibodies. To avoid the inclusion of whole viruses (either infectious or inactivated) within potential vaccines, researchers are increasingly looking to use engineered “virus-like particles” or VLPs, that provide the same particulate structure as a virus, but are non-infectious. However, this is usually performed by modifying individual viruses for each vaccine (i.e. a VLP for HIV, a different VLP for Ebola etc.). The inventors have therefore generated generic platforms for the production of VLPs that can contain viral glycoproteins from a wide range of different viruses. This versatility provides distinct advantages over current virus specific approaches. The present invention relies on the combination of two technical innovations, i.e. (i) core technology to generate VLPs, and (ii) technology to incorporate viral glycoproteins of the inventor's choice into the surface membrane of the engineered VLP.

The core technology to generate VLPs is based on the modification of the Mumps Virus matrix proteins to generate non-infectious VLPs. The Mumps virus matrix protein by itself is unable to form VLP. However, the incorporation of the membrane targeting sequence (MTS) leads to very efficient virus particle release. The MTS is derived from another protein known as Fyn-like protein kinase (19). Without wishing to be bound to any particular theory, the inventors believe that the use of this sequence in conjunction with the Mumps matrix protein with the express intention to generate VLPs is a non-obvious step. The inventors have shown that the matrix protein of a second closely related virus, Parainfluenza Virus 5 (PIV5), can be similarly modified by the same membrane targeting sequence to efficiently generate VLPs.

The technology for incorporating viral glycoproteins of choice into the generated VLPs, known as “pseudotyping” is mediated by fusing the external viral glycoprotein sequence of a chosen target glycoprotein (for examples HIV, Ebola, Rabies etc.) to the protein sequence of the Mumps viral glycoprotein that embeds (or inserts) itself within the viral particle, known as the “transmembrane domain”. This means that the external surface of the VLP exposes the external domain of the glycoprotein of choice but is tethered to the VLP by the inclusion of the common Mumps transmembrane domain and cytoplasmic tail. The inventors have shown that the transmembrane domains and cytoplasmic tail of Mumps and PIV5 can be interchanged for this purpose.

Whilst the fusion proteins of the present invention may comprise a TMD, this can be achieved by co-expression of any membrane protein that co-localises with the assembly of the matrix protein at the plasma membrane through passive incorporation into the budding VLP. This is generally applicable to any protein with a transmembrane domain, although typically viral, with or without a cytoplasmic tail. An example of this is the incorporation of HIV envelope protein (ConSOSL.UFO.750) into mumps matrix VLPs (FIG. 5A (VLP2) and FIG. 5B). A variant of this approach is to exchange the transmembrane domain of the WT envelope protein to that of a paramyxovirus. An example of this is shown diagrammatically in FIG. 1A. Experimental data to support this approach is shown in FIG. 5. Where that native protein does not naturally assemble with the matrix derived VLP, the swapping of WT transmembrane domain for that of the paramyxovirus matched to the matrix protein may be advantageous to maximise VLP incorporation. This approach can be readily applied to a wide range of viral glycoproteins such as Nipah virus, Rabies virus, SARS coronavirus, Lassa fever virus, and Ebola virus etc.

Nevertheless, and while not wishing to be bound to any particular theory, the approach is not limited to proteins that encode transmembrane domains and linkage of any protein to the glycoprotein transmembrane domain of paramyxovirus would result in incorporation into matrix derived VLPs.

The combination of these two steps allows for the generation of VLPs displaying multiple copies of the viral glycoprotein of our choice. These can be manufactured using mammalian cell culture platforms to generate VLPs that then form the vaccine for injection. Thus, the inventors are able to produce VLPs containing either the Mumps or PIV5 matrix proteins but displaying viral glycoproteins of choice, e.g. HIV or other viruses. When used as a vaccine this facilitates the induction of antibodies to the target vial glycoprotein. The inventors are also able to encode the required sequences as DNA or RNA vaccines that can then be injected as a vaccine to generate VLPs within the injected tissue (typically the skin or muscle), either with matrix and glycoprotein components delivered on separate constructs or the matrix and glycoprotein components delivered as a contiguous single sequence separated by a T2A cleavage sequence. This provides an alternative mechanism for delivering vaccine.

### REFERENCES

(1) Plotkin S A. Correlates of protection induced by vaccination. Clin Vaccine Immunol. 2010; 17:1055-1065.
(2) Lauring A S, Jones J O, Andino R. Rationalizing the development of live attenuated virus vaccines. Nat Biotechnol. 2010; 28:573-9.
(3) Berkhout B, Verhoef K, van Wamel J L, Back N K. Genetic instability of live, attenuated human immunodeficiency virus type 1 vaccine strains. J Virol.1999; 73:1138-45.
(4) Hastie, K. M., Zandonatti, M. A., Kleinfelter, L. M., Heinrich, M. L., Rowland, M. M., Chandran, K., Branco, L. M., Robinson, J. E., Garry, R. F., and Saphire, E. O. (2017). Structural basis for antibody-mediated neutralization of Lassa virus. Science 356, 923-928. (5) Klasse, P. J. (2012). The molecular basis of HIV entry. Cell Microbiol 14, 1183-1192.
(6) Lee, J., Nyenhuis, D. A., Nelson, E. A., Cafiso, D. S., White, J. M., and Tamm, L. K. (2017). Structure of the Ebola virus envelope protein MPER/T M domain and its interaction with the fusion loop explains their fusion activity. Proc Natl Acad Sci USA 114, E7987-E7996.
(7) Sarkar, A., Bale, S., Behrens, A. J., Kumar, S., Sharma, S. K., de Val, N., Pallesen, J., Irimia, A., Diwanji, D. C., Stanfield, R. L., et al. (2018). Structure of a cleavage-independent HIV Env recapitulates the glycoprotein architecture of the native cleaved trimer. Nat Commun 9, 1956.
(8) Sanders, R. W., and Moore, J. P. (2017). Native-like Env trimers as a platform for HIV-1 vaccine design. Immunol Rev 275, 161-182.
(9) Gao, Y., Wijewardhana, C., and Mann, J. F. S. (2018). Virus-Like Particle, Liposome, and Polymeric Particle-Based Vaccines against HIV-1. Front Immunol 9, 345.
(10) Mohan, T., Berman, Z., Luo, Y., Wang, C., Wang, S., Compans, R. W., and Wang, B. Z. (2017). Chimeric virus-like particles containing influenza H A antigen and GPI-CCL28 induce long-lasting mucosal immunity against H3N2 viruses. Sci Rep 7, 40226.
(11) Storcksdieck genannt Bonsmann, M., Niezold, T., Temchura, V., Pissani, F., Ehrhardt, K., Brown, E. P., Osei-Owusu, N. Y., Hannaman, D., Hengel, H., Ackerman, M. E., et al. (2015). Enhancing the Quality of Antibodies to HIV-1 Envelope by GagPol-Specific Th Cells. J Immunol 195, 4861-4872.
(12) Ramirez, A., Morris, S., Maucourant, S., D'Ascanio, I., Crescente, V., Lu, I. N., Farinelle, S., Muller, C. P., Whelan, M., and Rosenberg, W. (2018). A virus-like particle vaccine candidate for influenza A virus based on multiple conserved antigens presented on hepatitis B tandem core particles. Vaccine 36, 873-880.
(13) Elsayed, H., Nabi, G., McKinstry, W. J., Khoo, K. K., Mak, J., Salazar, A. M., Tenbusch, M., Temchura, V., and Uberla, K. (2018). Intrastructural Help: Harnessing T-Helper Cells Induced by licensed Vaccines for Improvement of HIV Env Antibody Responses to Virus-Like Particle Vaccines. J Virol.
(14) Zhu, P., Liu, J., Bess, J., Jr., Chertova, E., Lifson, J. D., Grise, H., Ofek, G. A., Taylor, K A., and Roux, K. H. (2006). Distribution and three-dimensional structure of AIDS virus envelope spikes. Nature 441, 847-852.
(15) L, M., Schmitt, P. T., Li, Z., McCrory, T. S., He, B., and Schmitt, A. P. (2009). Mumps virus matrix, fusion, and nucleocapsid proteins cooperate for efficient production of virus-like particles. J Virol 83, 7261-7272.
(16) Terrier, O., Rolland, J. P., Rosa-Calatrava, M., Lina, B., Thomas, D., and Moules, V. (2009). Parainfluenza virus type 5 (PIV-5) morphology revealed by cryo-electron microscopy. Virus Res 142, 200-203.
(17) Ludwig, K., Schade, B., Bottcher, C., Korte, T., Ohlwein, N., Baljinnyam, B., Veit, M., and Herrmann, A. (2008). Electron cryomicroscopy reveals different F1+F2 protein States in intact parainfluenza virions. J Virol 82, 3775-3781.
(18) Cox, R. M., and Plemper, R. K. (2017). Structure and organization of paramyxovirus particles. Curr Opin Virol 24, 105-114.
(19) Wang D, Harmon A, Jin J, Francis D H, Christopher-Hennings J, Nelson E, Montelaro R C, Li F. The lack of an inherent membrane targeting signal is responsible for the failure of the matrix (M1) protein of influenza A virus to bud into virus-like particles. J Virol. 2010 Mav:84(9):4673-81.

1. A fusion protein comprising an antigen, and a Paramyxovirus or Orthomyxovirus transmembrane domain (TMD) and/or a Paramyxovirus or Orthomyxovirus cytoplasmic tail (CT).

2. The fusion protein according to claim 1, wherein the antigen is a viral antigen, and wherein the TMD and/or CT is derived from a different virus from that of the viral antigen.

3. The fusion protein according to claim 1, wherein the antigen is derived from an envelope virus selected from the group consisting of: Retroviridae; Togaviridae; Arenaviridae; Flaviviridae; Orthomyxoviridae; Paramyxoviridae; Bunyaviridae; Rhabdoviridae; Filoviridae; Coronaviridae; Bornaviridae; and Arteriviridae.

4. The fusion protein according to claim 1, wherein the Paramyxovirus is selected from the group consisting of: Rubulavirus; Parainfluenzavirus 5; Parainfluenzavirus 2; Parainfluenzavirus 3; Respirovirus; Morbillivirus; Henipavirus; Avulavirus; Pneumovirus; and Metapneumovirus and/or the orthomyxovirus is be selected from the group consisting of: influenza virus A; influenza virus B; and influenza virus C.

5. The fusion protein according to claim 1, wherein the fusion protein comprises a viral antigen and a Parainfluenzavirus 5 or Rubulavirus TMD and a Parainfluenzavirus 5 or Rubulavirus CT.

6. The fusion protein according to claim 1, wherein the fusion protein comprises an amino acid sequence substantially as set out in SEQ ID NO: 5, or a biologically active variant or fragment thereof, or is encoded by a nucleic acid comprising a nucleotide sequence substantially as set out in SEQ ID NO: 6, or a variant or fragment thereof.

7. The fusion protein according to claim 1, wherein the fusion protein comprises an amino acid sequence substantially as set out in SEQ ID NO: 7, or a biologically active variant or fragment thereof, or is encoded by a nucleic acid comprising a nucleotide sequence substantially as set out in SEQ ID NO: 8, or a variant or fragment thereof.

8. A fusion protein suitable for forming a virus like particle (VLP), the fusion protein comprising a Paramyxovirus or Orthomyxovirus matrix protein and a membrane targeting signal (MTS).

9. The fusion protein according to claim 8, wherein the matrix protein is a paramyxovirus matrix protein selected from the group consisting of: Rubulavirus; Parainfluenzavirus 5; Parainfluenzavirus 2; Parainfluenzavirus 3; Respirovirus; Morbillivirus; Henipavirus; Avulavirus; Pneumovirus; and Metapneumovirus, or the matrix protein is a Orthomyxovirus matrix protein and is selected from the group consisting of: influenza virus A; influenza virus B; and influenza virus C.

10. The fusion protein according to claim 8, wherein the MTS is selected from the group consisting of: SEQ ID No: 9; SEQ ID No: 10, SEQ ID No: 11 and SEQ ID No: 12, or a variant or fragment thereof.

11. The fusion protein according to claim 8, wherein the fusion protein comprises an amino acid sequence substantially as set out in SEQ ID NO: 13, or a variant or fragment thereof, or is encoded by a nucleic acid comprising a nucleotide sequence substantially as set out in SEQ ID NO: 14, 15 or 16, or a variant or fragment thereof.

12. The fusion protein according to claim 8, wherein the fusion protein comprises an amino acid sequence substantially as set out in SEQ ID No: 17, or a biologically active variant or fragment thereof, or is encoded by a nucleic acid comprising a nucleotide sequence substantially as set out in SEQ ID NO: 18, 19 or 20, or a variant or fragment thereof.

13. A virus like particle (VLP) comprising the fusion protein according to claim 8.

14. A method of producing a virus like particle (VLP) according to the claim 13, the method comprising expressing a nucleic acid encoding the fusion protein of claim 8 in a host cell.

15-31. (canceled)
